(19) United States
(12) Patent Application Publication
(10) Pub. No.: US 2021/0063420 A1
(43) Pub. Date: Mar. 4, 2021

(54) Human Neural Organoid Platform for Detecting Drugs of Abuse Disorder Susceptibility and Therapeutic Countermeasure and Non-Addictive Pain Medication Development

(71) Applicant: Rene Anand, Westerville, OH (US)

(72) Inventors: Rene Anand, Westerville, OH (US); Susan McKay, Columbus, OH (US)

(21) Appl. No.: 16/999,656

(22) Filed: Aug. 21, 2020

Related U.S. Application Data
(60) Provisional application No. 62/889,853, filed on Aug. 21, 2019.

(51) Int. Cl. G01N 33/94

(52) U.S. Cl. CPC G01N 33/94; A61K 45/06

(57) ABSTRACT

Methods for using gene expression changes in response to drugs of abuse in human neural organoids to identify and predict susceptibility to drug abuse and develop therapeutic countermeasures are disclosed.

### CROSS-REFERENCE TO RELATED APPLICATION

This application claims priority to Provisional U.S. patent application Ser. No. 62/889,853, filed on Aug. 21, 2019, which is incorporated by reference in its entirety.

### FIELD OF THE INVENTION

This disclosure relates to production and use of human stem cell derived neural organoids for detecting susceptibility to drug addiction to multiple drugs of abuse in a human, using a patient-specific pharmacotherapy strategies. Further disclosed are patient-specific pharmacotherapeutic methods for reducing addiction risk in a human. Also disclosed are methods to predict the risk of addiction in an individual. In particular, the inventive processes disclosed herein provide neural organoid reagents produced from an individual's induced pluripotent stem cells (iPSCs) for identifying patient-specific pharmacotherapy, predictive biomarkers, and developmental and pathogenic gene expression patterns, and dysregulation thereof in addiction onset and progression, and methods for diagnosing prospective and concurrent risk of addiction in the individual. The invention also provides reagents and methods for identifying, testing, and validating therapeutic modalities, including chemical and biologic molecules for use as drugs for ameliorating or curing addiction.

### BACKGROUND OF THE INVENTION

There is significant evidence from epidemiological studies that prenatal exposure to nicotine adversely affect the developing human fetus resulting in adverse effects on human behavioral health. Nicotine crosses the placental barrier easily and its concentration in fetal serum is higher than in maternal serum (Dempsey and Benowitz, 2001 Drug Saf.; 24: 277-322).

In addition, there is evidence that nicotine is a ‘gateway’ drug for addiction to other drugs of abuse including cocaine. NIDA. (“NIH study examines nicotine as a gateway drug.” National Institute on Drug Abuse, 2 Nov. 2011, https://archives.drugabuse.gov/news-events/news-releases/2011/11/nih-study-examines-nicotine-gateway-drug).

Thus, there remains a critical need to understand the effects of nicotine on the developing human brain. Most of these effects have been inferred from rodent studies, or more recently, at the functional level using MRI of adolescent children of mothers who used drugs during pregnancy (Rando et al., 2013 Biol Psychiatry., 74: 482-489).

Additionally, genome-wide association studies are rapidly revealing a multitude of genetic variants that increase susceptibility to tobacco abuse disorders (Breitling et al. 2009; Wessel et al. 2010 Neuropsychopharmacology, 35: 2392-402; Han et al. 2011 Am J Med Genet B Neuropsychiatr Genet., 156B: 421-9; Xie et al. 2011 Biol Psychiatry, 70(6): 528-36; Bierut et al. 2007 Hum Mol Genet., 16: 24-35; Baker et al. 2009 Nicotine Tob Res. 11: 785-96; Greenbaum et al. 2006 Mol Psychiatry., 11: 312-22; Saccone et al. 2007 Hum Mol Genet. 16: 36-49; Zeiger et al. 2008 Hum Mol Genet. 17: 724-34; Hoft et al. 2009 Neuropsychopharmacology, 34: 698-706.; Berrettini et al. 2008 Mol Psychiatry. 13: 368-73; Bierut et al. 2007; Caporaso et al. 2009, PLoS One, 4: e4653. Saccone et al. 2007; Thorgeirsson and Stefansson 2008 Biol Psychiatry, 64: 919-21; Thorgeirsson et al. 2010, Nat Genet., 42: 448-53.).

Thus, there remains a critical need to understand the effects of nicotine on human brain development in utero and subsequent priming of the adult human brain for drug abuse including cocaine, heroin, opioids, marijuana and tobacco and nicotine itself.

The development of the mDA neurons is highly regulated by both extrinsic and intrinsic factors. Early patterning, fate determination and specification of mDA neurons are mediated by extrinsic signals including sonic hedgehog (shh), fibroblast growth factor 8 (fgf8), transforming growth factor β (TGF-β) and wingless (Wnt), which in turn activate transcription factors such as Lmx1a, Lmx1b, Neurog2, En1, En2, Pitx3 and Nurr1. Mice deficient in Pitx3, Lmx1b, En1, En2, Nurr1 and TGF-β have played a key role in elucidating these genes involved in mDA neuron development (Smidt et al., 2004 Development.131: 1145-55.; Saucedo-Cardenas et al., 1998 PNAS. 95(7): 4013-8.; Smidt et al., 2004; Simon et al., 2004 Cell Tissue Res. 318: 53-61.). Lmbx1a and 1b are early factors involved in mDA neuron differentiation: Lmx1a activates Msx1, and together they activate Ngn 2, which, in turn, activates Sox2-positive progenitors that later give rise to Nurr1 expressing post mitotic DA neurons. Nurr1 regulates the expression of genes for dopaminergic neurons including tyrosine hydroxylase, dopamine transporter, vesicular monamine transporter 2, and Ret receptor kinase (Saucedo-Cardenas et al., 1998 PNAS. 95(7): 4013-8., Smits et al., 2003 Eur J Neurosci. 18: 1731-8.; Wallén et al., 2001 Mol Cell Neurosci. 18: 649-63, Zetterstrom et al., 1997 Science 276: 248-50.) and nAChR subunits α6 and β3 (Chakrabarty et al., 2012 Biol Open. 1: 693-704). The En genes are required for maintaining mDA neurons (Simon et al., 2004 Cell Tissue Res. 318: 53-61., Alberi et al., 2005; Simon et al., 2003 Ann N Y Acad Sci. 991: 36-47.; Simon et al., 2005 Mol Cell Neurosci. 28(1): 96-105.; Thuret et al., 2004; Sgadó et al.,2006 PNAS. 103: 15242-15247. 2006). Sox6, OTX2, Noz1 and NDNF define subpopulations of mDA neurons (Panman et al., 2014 Cell Rep., 8(4): 1018-25.; Poulin et al., 2014 Cell Rep. Nov 6; 9(3): 930-43).

Nicotine acts on a multitude of nAChR subtypes assembled from α1-α7 and β2-β3 subunits. In the embryonic brain, GABA serves to elicit depolarizing, excitatory responses (Rivera et al., 1999; Ben-Ari, 2002 Nat Rev Neurosci. 3:728-39.). The switch from GABA-mediated excitation to inhibition is mediated by the α7 nAChR whose loss delays maturation of chloride gradients suggesting sequential interplay of nAChR mediated signaling and GABAergic signaling guides neuronal development (Liu et al., 2006 Science, 3141610-314163.) and that nicotine could alter excitation-inhibition homeostasis by delaying this switch over. In the adult human brain, nicotine exposure affects many areas expressing nAChRs to increase susceptibility to neuropsychiatric and addiction disorders. In rodents, nicotine modulates several behaviors including 1) impulse control and attention by acting in the prefrontal cortex (Goriounova and Mansvelder, 2012 J Neurosci. 32:10484-93), 2) reward salience via regulation of the VTA-striatum circuitry and 3) aversive salience by regulating the medial habenula-interpenducular nucleus circuitry. Specifically, DA neurons in the VTA are locally inhibited by GABAergic interneurons expressing α4β2 nAChRs (Mansvelder et al., 2002 Neuron, 33: 905-19; Tolu et al., 2013 Mol Psychiatry, 18: 382-93.). In contrast, excitatory glutamatergic inputs of the lateral dorsal tegmental and pontine peduncular tegmental area shift tonic firing of VTA dopaminergic neurons to burst firing to encode reward or aversion salience (Lammel et al., 2012, Nature, 491: 212-217.).

Multiple nAChR subtypes are important for nicotine addiction: the α4β2 nAChRs in VTA projections to the NAc (Picciotto et al., 1998 Nature; 391173-7; Tapper et al., 2004 Science. 306: 1029-1032); the α6α4β2β3 and α6β2β3 in SNpc projections to the striatum (Quik et al., 2011 Biochem Pharmacol., 82: 873-82.), and the α3α5β4 and/or α4α5β2 in medial habenula projections to the interpeduncular nucleus (Fowler et al., 2011 Nature, 471: 597-601). These nAChRs exhibit significantly different channel kinetics, rates of desensitization and affinities for ACh and nicotine. Most of these nAChRs are expressed in DAergic, glutamatergic and GABAergic neurons and modulate pre-synaptic dopamine, GABA and glutamate release probability (Wonnacott 1997 Trends Neurosci., 20: 92-8) but some are also expressed on the soma. Repeated nicotine exposure also activates intracellular signaling pathways that alter expression of genes regulating dendrite and spine structure (Lozada et al., 2012 J Neurosci. 32: 8391-400.) as well as expression of miRNAs that post-transcriptionally regulate these processes (Lippi et al., 2011 J Cell Biol., 194: 889-904), thus fundamentally changing the neuro architecture of the mDA circuits (Russo et al., 2010 Trends Neurosci. 33: 267-277; Kalivas et al., 2009 Neuropharmacology., 56, (Suppl 1): 169-173; Wolf, 2010 Neurotox Res., 18: 393-409).

Nicotine, in addition to acting as an agonist, also acts as an intracellular chemical chaperone at nM concentrations to catalyze subunit assembly in the endoplasmic reticulum. This results in the upregulation of both α4β2 nAChRs (Sallette et al., 2005 Neuron. 46(4): 595-607; Kuryatov et al., 2005 Mol Pharmacol., 68: 1839-51; Lester et al., 2009 AAPS J., 11(1): 167-77) and a6* nAChRs (Walsh et al., 2008 J Biol Chem., 283: 6022-32) and this is the main mechanism by which nicotine upregulates nAChRs in the brains of humans, rodents and primates (Breese et al., 1997 J Pharmacol Exp Ther., 282: 7-13.; Mamede et al., 2007 J Nucl Med., 48: 1829-35; Marks et al., 1998 J Pharmacol Exp Ther. 285: 377-86; Nashmi et al., 2007 J Neurosci. 27: 8202-18.; Schwartz et al., 1983 Science. 220: 214-6).

Our data indicate that chronic nicotine exposure of human iPSC-derived brain organoids increases the expression of a set of genes for proteins that are involved in the development of midbrain dopaminergic neurons (Blaess and Ang, 2015, Rev Dev Biol. Jan 6) and especially the ventral tegmental area (VTA) and substantia nigra pars compacta (SNpc). These include the expression of genes encoding transcription factors and coactivators or repressors whose temporal and spatial expression helps develop the midbrain region. The primary mediators of the underlying receptors by which nicotine exposure alters the expression of these genes to promote midbrain development and function at different doses of nicotine in addicts at steady state (50-100 nM) and peak (0.5-1 μM) concentrations of nicotine in the serum provides biomarkers as antidote targets for multiple drugs including opioids and pain medication addiction (Oxycontin, Fentanyl as exemplars).

Improved experimental models of the human brain are urgently required to understand the susceptibility for drug abuse and test potential therapeutics. The ability to detect drug abuse susceptibility in the early stages could prove critical in the effective management of substance abuse and development of therapeutic countermeasures. Consistent with this molecular diagnostics promises to provide a basis for early detection and a risk of early onset of neurological disease. However, molecular diagnostic methods are limited in accuracy, specificity, and sensitivity. Therefore, there is a need in the art for non-invasive, patient specific molecular diagnostic methods to be developed.

### SUMMARY OF THE INVENTION

This disclosure, in one embodiment, provides neural reagents and methods for identifying or treating drug abuse susceptibility in a human, using patient-specific pharmacotherapies, the methods comprising: procuring one or a plurality of cell samples from a human, comprising one or a plurality of cell types; reprogramming the one or the plurality of cell samples to produce one or a plurality of induced pluripotent stem cell samples; treating the one or the plurality of induced pluripotent stem cell samples to obtain one or more patient specific neural organoids; collecting a biological sample from the patient specific neural organoid; detecting changes in drug abuse susceptibility biomarker expression from the patient specific neural organoid sample that are differentially expressed in humans who abuse drugs or have susceptibility to do so; performing assays on the patient specific neural organoid to identify therapeutic agents that alter the differentially expressed drug abuse susceptibility biomarkers in the patient-specific neural organoid sample; and administering therapeutic agents for drug abuse susceptibility to treat the human. In one aspect, at least one cell sample reprogrammed to the induced pluripotent stem cell phenotype is a human skin or blood cell derived from a fibroblast derived from skin or blood cells from humans. In another aspect the fibroblast-derived skin or blood cells from humans is identified with the genes identified in Table 1 (Novel Drug Abuse Susceptibility Biomarkers) or Table 4 (Therapeutic Drug Abuse Susceptibility Biomarkers). The nucleotide sequence of genes identified in the included tables as well as genes associated with fibroblasts derived from skin or blood cells can be found in publicly available databases such as Genecards, Genbank, and Pubmed, NIH (dbGaP. ClinVar, dbSNP), and National Institute of Drug Abuse (NIDA) among others. In yet another aspect, the measured biomarkers comprise nucleic acids, proteins, or their metabolites. In another aspect, the measured biomarkers comprise one or a plurality of biomarkers identified in Table 1 or Table 4 or variants thereof. In yet another aspect, a combination of drug abuse susceptibility biomarkers is detected, the combination comprising a nucleic acid encoding human NURR1, Lmx1a, Lmx1b, Neurog2, OTX2, Nolz1, and NDN F, associated variants and one ora plurality of biomarkers comprising a nucleic acid encoding human genes identified in Table 1.

In one aspect of the disclosure, the biomarkers for drug abuse susceptibility include human nucleic acids, proteins, or their metabolites as listed in Table 1.

TABLE 1
Novel Drug Abuse Susceptibility Biomarkers

| Gene | Multi-Drug Use Susceptibility |
|---|---|
| ADRBK2 | Cocaine Abuse |
| ATP13A4 | Tobacco Smoking |
| ATP1A2 | Tobacco Smoking |
| C14orf28 | Dopamine Receptor Interacting Protein-1 |
| DRD2 | Cocaine Dependence. |
| DUSP6 | Pain; Dual Specificity Phosphatase 6 alleviating |
| | chronic postoperative pain |
| FAAH | Polysubstance Abuse; Cannabis Dependence. |
| GLRA1 | Glycine receptor; Neuropathic pain Hyperekplexia 1 |
| | and Hyperekplexia. |
| GLRB | Glycine receptor; Neuropathic pain, Hyperekplexia 2 |
| | and Hyperekplexia. |
| GNAO1 | Drug Cue induced craving |
| GNAQ | Drug Cue induced craving |
| HIF3A | Tobacco Smoking |
| IGFBP7 | Tobacco Smoking |
| NR4A1 | Opioid addiction and microglia protein |
| NR4A2 | Opioid addiction |
| OPRK1 | Morphine Dependence Alcohol Dependence. |
| OSBPL1A | Cocaine and Heroin |
| PLOD2 | Tobacco Smoking |
| PREX2 | Tobacco Smoking |
| PTN | Tobacco Smoking |
| RGS17 | Substance Dependence. |
| RGS20 | Negatively regulates mu-opioid receptor-mediated |
| | activation of the G-proteins |
| S1PR1 | Tobacco Smoking |
| SLC1A2 | Tobacco Smoking |
| SLC1A3 | Tobacco Smoking |
| SLC3A2 | Tobacco Smoking |
| SLC4A4 | Tobacco Smoking |
| SLC7A11 | Tobacco Smoking |
| SOX2 | Heroin |
| SV2A | Alcohol-Related Birth Defect |
| SYT1 | Cocaine and Heroin |
| TAC1 | Heroin |
| TH | Conversion of tyrosine to dopamine |
| TIMP1 | Cocaine and heroin |
| TP53BP2 | Tobacco Smoking |
| TRIM59 | Drug cue induced |
| VEGFA | Tobacco Smoking |
| ZBTB16 | Tobacco Smoking |

One of skill in the art will recognize that sequence data for the genes listed above can be obtained in publicly available gene databases such as GeneCards, GenBank, Malcard, Uniport and PathCard databases.

In still another aspect, the neural organoid biological sample is collected after about one hour up to about 12 weeks post inducement. In another aspect, the neural organoid sample is procured from structures of the neural organoid that mimic structures developed in utero at about 5 weeks. In yet another aspect the neural organoid at about twelve weeks post-inducement comprises structures and cell types of retina, cortex, midbrain, hindbrain, brain stem, or spinal cord. In a one aspect, the neural organoid contains microglia, and one or a plurality of drug abuse susceptibility biomarkers as identified in Table 1.

In yet another aspect the method is used to detect environmental factor susceptibility including infectious agents that cause or exacerbate tumors and cancer, or accelerators of tumor and cancer growth and metastasis. In a further aspect, the method is used to identify nutritional factor deficiency susceptibility or supplements for treating tumors and cancer. In a further aspect, genes identified in Tables 1 or 4 for nutritional factors related to pathways (Pathcards database; Weizmann Institute of Science) regulate the nutritional factor or supplement. In yet another aspect, fetal cells from amniotic fluid can be used to grow neural organoids and as such, nutritional and toxicological care can begin even before birth so that the child develops in utero well.

In another aspect, the measured biomarkers comprise biomarkers identified in Table 1 or Table 4 can be nucleic acids, proteins, or their metabolites (identifiable in GeneCard, Genbank, Pubmed, and PathCard databases). In a further aspect the invention provides diagnostic methods for predicting drug abuse susceptibility in a human, comprising one, a plurality subset of the biomarkers as identified in Table 1, or Table 4 with corresponding sequences found in publicly available databases such as Genecards, Genbank, and Pubmed among others. In yet another aspect, the subset of measured biomarkers comprise nucleic acids, proteins, or their metabolites as identified in Table 1 or Table 4 with corresponding sequences found in publicly available databases such as Genecards, Genbank, and Pubmed among others. The biomarkers can be correlated to drug abuse susceptibility.

In another embodiment are methods of pharmaceutical testing for drug abuse susceptibility drug screening, toxicity, safety, and/or pharmaceutical efficacy studies using patient-specific neural organoids.

In an additional embodiment, methods are provided for detecting at least one biomarker of drug abuse susceptibility, the method comprising, obtaining a biological sample from a human patient; and contacting the biological sample with an array comprising specific-binding molecules for the at least one biomarker and detecting binding between the at least one biomarker and the specific binding molecules.

In one aspect, the biomarker detected is a gene therapy target.

In a further embodiment, the disclosure provides a kit comprising an array containing sequences of biomarkers from Table 1 for use in a human patient. In one aspect, the kit further contains reagents for RNA isolation and biomarkers for drug abuse susceptibility. In a further aspect, the kit advantageously comprises a container and a label or instructions for collection of a sample from a human, isolation of cells, inducement of cells to become pluripotent stem cells, growth of patient-specific neural organoids, isolation of RNA, execution of the array and calculation of gene expression change and prediction of concurrent or future disease risk. In one aspect of the disclosure, the biomarkers for drug abuse susceptibility include human nucleic acids, proteins, or their metabolites as listed in Table 1.

In another aspect, biomarkers can comprise any markers or combination of markers in Tables 1, 4, or 5 or variants thereof.

One of skill in the art will recognize that sequence data for the genes listed above can be obtained in publicly available gene databases such as GeneCards, GenBank, Malcard, Uniport and PathCard databases.

In a further embodiment, the disclosure provides a method for detecting one or a plurality of biomarkers from different human chromosomes associated with drug abuse susceptibility using data analytics that obviates the need for whole genome sequence analysis of an individual's genome. In one aspect, the methods are used to determine gene expression level changes that are used to identify clinically relevant symptoms and treatments. In yet another aspect, the neural organoids are used to identify novel biomarkers that serve as data input for development of algorithm techniques as predictive analytics. In a further aspect the algorithmic techniques include artificial intelligence, machine and deep learning as predictive analytics tools for identifying biomarkers for diagnostic, therapeutic target and drug development process for drug abuse and drug abuse susceptibility. In one aspect, the neural organoid along with confirmatory data and novel data can be used to develop signature algorithms with machine learning, artificial intelligence and deep learning. In another aspect, the method is used for diagnostic, therapeutic target discovery and drug action discovery for drug abuse susceptibility. In yet another aspect, the inventive model neural organoid data is corroborated by analysis of tissues from drug abuse patients and extensively identifies known biomarkers for drug abuse. In yet another aspect the method is used with induced pluripotent stem cells from any skin cell, tissue, or organ from the human body allowing for an all-encompassing utility for diagnostics, therapeutic target discovery, and drug development.

In yet another embodiment the invention provides methods for predicting a risk of drug abuse susceptibility. Said methods first determine gene expression changes in neural organoids from a normal human individual versus a human individual at risk of drug abuse. Genes that change greater than 1.4 fold are associated with drug abuse susceptibility as understood by those skilled in the art.

In a further embodiment, the invention provides kits for predicting the risk of drug abuse and drug abuse susceptibility. Said kits provide reagents and methods for identifying from a patient sample gene expression changes for one or a plurality of disease-informative genes for individuals that do not abuse drugs.

In an additional embodiment, the invention provides methods for identifying therapeutic agents for treating drug abuse. Such embodiments comprise using the neural organoids provided herein, particularly, but not limited to said neural organoids from iPSCs from an individual or from a plurality or population of individuals. The inventive methods include assays on said neural organoids to identify therapeutic agents that alter drug abuse related changes in expression of genes identified as having altered expression patterns in disease, as to restore gene expression of disease-informative gene patterns to more closely resemble the expression patterns in individuals without drug abuse susceptibility.

In another embodiment, the invention provides methods for predicting a risk for developing drug abuse susceptibility or drug abuse in a human, comprising procuring one or a plurality of cell samples from a human, comprising one or a plurality of cell types; reprogramming the one or the plurality of cell samples to produce one or a plurality of induced pluripotent stem cell samples; treating the one or the plurality of induced pluripotent stem cell samples to obtain one or more patient specific neural organoids; collecting a biological sample from the patient specific neural organoid; measuring biomarkers in the neural organoid sample; and detecting measured biomarkers from the neural organoid sample that are differentially expressed in humans abusing drugs. In certain embodiments, at least one cell sample reprogrammed to the induced pluripotent stem cell is a fibroblast. In certain embodiments, the measured drug abuse susceptibility biomarkers comprise nucleic acids, proteins, or their metabolites. In certain embodiments, the measured biomarker is a nucleic acid encoding human NURR1, Lmx1a, Lmx1b, Neurog2, OTX2, Nolz1, and NDNF and associated variants. In certain embodiments, the measured biomarkers comprise one or a plurality of genes as identified in Tables 1, 4, or 5 . In certain embodiments, the neural organoid sample is procured from minutes to hours up to 15 weeks post inducement. In certain embodiments, the biomarkers to be tested are one or a plurality of biomarkers in Tables 1, 4, or 5.

In another embodiment is disclosed a method of using a neural organoid along with confirmatory data, and novel data to develop signature algorithms with machine learning, artificial intelligence and deep learning for drug abuse susceptibility. The can be used for diagnostic, therapeutic target discovery and drug action discovery for drug abuse and drug abuse related comorbidities as listed in Table 6.

In a further aspect, the neural inventive novel organoid data is corroborated in post mortem or biopsy tissues from idiopathic patients and extensively identifies known biomarkers for the susceptibility to drug abuse and comorbidities. In a further aspect, the method is used with induced pluripotent stem cells from any skin cell, tissue, or organ from the human body allowing for an all-encompassing utility for diagnostics, therapeutic target discovery, and drug development. The method is further advantageous in that the disclosed method and/or neural organoid is used for guided and patient specific toxicology guided by genes form patient's selective vulnerability to infectious agents or to accumulate currently EPA approved safe levels of copper. In a further aspect, the method can be used to identify nutritional and toxicological care that can begin even before birth so that the child develops normally in utero. Further, the disclosed method in neural organoids can be used to obtain human exosomes for diagnostic and therapeutic purposes of brain disease. The measured biomarkers comprise nucleic acids, proteins, or their metabolites such as cholesterol.

The method disclosed is intended to help identify individuals with a risk of being susceptible to abusing addictive opioid medications including OxyContin and fentanyl. The method can also be used to identify the risk of developing the comorbidities of cancer, perturbation of circadian rhythms, and neuropsychiatric disorders, including schizophrenia in individuals with drug abuse susceptibility or the co-morbidities listed in Table 6.

In a further aspect, the neural organoid model can be used to identify novel non-addictive pain medications.

### BRIEF DESCRIPTION OF THE FIGURES

FIG. 1A is a micrograph showing a 4X dark field image of Brain Organoid Structures typical of approximately 5-week in utero development achieved in 12 weeks in vitro. Average size: 2-3 mm long. A brain atlas is provided for reference (left side).

FIG. 1B shows immuno-fluorescence images of sections of iPSC-derived human brain organoid after approximately 12 weeks in culture. Z-stack of thirty-three optical sections, 0.3 microns thick were obtained using laser confocal imaging with a 40× lens. Stained with Top panel: beta III tubulin (green: axons); MAP2 (red: dendrites); Hoechst (blue: nuclei); Bottom panel: Doublecortin (red).

FIG. 2 is a micrograph showing immunohistochemical staining of brain organoid section with the midbrain marker tyrosine hydroxylase. Paraformaldehyde fixed sections of a 8-week old brain organoid was stained with an antibody to tyrosine hydroxylase and detected with Alexa 488 conjugated secondary Abs (green) and counter stained with Hoechst to mark cell nuclei (blue). Spinning disc confocal image (40× lens) of section stained with an antibody that binds tyrosine hydroxylase and Hoechst (scale bar: 10 μm).

FIG. 3 is a spinning disc confocal image (40× lens) of brain section. Astrocytes stained with GFAP (red) and mature neurons with NeuN (green).

FIG. 4 is a schematic showing in the upper panel a Developmental Expression Profile for transcripts as Heat Maps of NKCC 1 and KCC2 expression at week 1, 4 and 12 of organoid culture as compared to approximate known profiles (lower panel). NKCCI: Na(+)-K(+)-Cl(−) cotransporter isoform 1. KCC2: K(+)-Cl(−) cotransporter isoform 2.

FIG. 5A is a schematic showing GABAergic chloride gradient regulation by NKCC 1 and KCC2.

FIG. 5B provides a table showing a representative part of the entire transcriptomic profile of brain organoids in culture for 12 weeks measured using a transcriptome sequencing approach that is commercially available (AmpliSeg™). The table highlights the expression of neuronal markers for diverse populations of neurons and other cell types that are comparable to those expressed in an adult human brain reference (HBR; Clontech) and the publicly available embryonic human brain (BRAINS CAN) atlas of the Allen Institute database.

FIG. 5C provides a table showing AmpliSeq™ gene expression data comparing gene expression in an organoid (column 2) at 12 weeks in vitro versus Human Brain Reference (HBR; column 3). A concordance of greater than 98% was observed.

FIG. 5D provides a table showing AmpliSeg™ gene expression data comparing organoids generated during two independent experiments after 12 weeks in culture (column 2 and 3). Gene expression reproducibility between the two organoids was greater than 99%. Note that values are CPM (Counts Per Kilo Base per Million reads) in the tables and <1 is background.

FIG. 6A is a schematic showing results of developmental transcriptomics. Brain organoid development in vitro follows KNOWN Boolean logic for the expression pattern of transcription factors during initiation of developmental programs of the brain. Time Points: 1, 4, and 12 Weeks. PITX3 and NURRI (NR4A) are transcription factors that initiate midbrain development (early; at week 1), DLKI, KLHLI, PTPRU, and ADH2 respond to these two transcription factors to further promote midbrain development (mid; at week 4 &12), and TH, VMAT2, DAT and D2R define dopamine neuron functions mimicking in vivo development expression patterns. The organoid expresses genes previously known to be involved in the development of dopaminergic neurons (Blaess S, Ang SL. Genetic control of midbrain dopaminergic neuron development. Wiley Interdiscip Rev Dev Biol. 2015 Jan. 6. doi: 10.1002/wdev. 169).

FIGS. 6B-6D are tables showing AmpliSeg™ gene expression data for genes not expressed in organoid (column 2 in 6B, 6C, and 6D) and Human Brain Reference (column 3 in 6B, 6C, and 6D). This data indicates that the organoids generated do not express genes that are characteristic of non-neural tissues. This gene expression concordance is less than 5% for approximately 800 genes that are considered highly enriched or specifically expressed in a non-neural tissue. The olfactory receptor genes expressed in the olfactory epithelium shown are a representative example. Gene expression for most genes in table is less than one or zero.

FIG. 7 includes schematics showing developmental heat maps of transcription factors (TF) expressed in cerebellum development and of specific Markers GRID 2.

FIG. 8 provides a schematic and a developmental heat map of transcription factors expressed in Hippocampus Dentate Gyms.

FIG. 9 provides a schematic and a developmental heat map of transcription factors expressed in GABAergic Interneuron Development. GABAergic Interneurons develop late in vitro.

FIG. 10 provides a schematic and a developmental heat map of transcription factors expressed in Serotonergic Raphe Nucleus Markers of the Pons.

FIG. 11 provides a schematic and a developmental heat map of transcription factor transcriptomics (FIG. 11A). Hox genes involved in spinal cord cervical, thoracic, and lumbar region segmentation are expressed at discrete times in utero. The expression pattern of these Hox gene in organoids as a function of in vitro developmental time (1 week; 4 weeks; 12 weeks; ; FIGS. 11B and 11C)

FIG. 12 is a graph showing the replicability of brain organoid development from two independent experiments. Transcriptomic results were obtained by Ampliseq analysis of normal 12-week old brain organoids. The coefficient of determination was 0.6539.

FIG. 13 provides a schematic and gene expression quantification of markers for astrocytes, oligodendrocytes, microglia, and vasculature cells.

FIG. 14 shows developmental heat maps of transcription factors (TF) expressed in retina development and other specific Markers. Retinal markers are described, for example, in Farkas et al. 2013 BMC Genomics, 14:486.

FIG. 15 shows developmental heat maps of transcription factors (TF) and Markers expressed in radial glial cells and neurons of the cortex during development

FIG. 16 is a schematic showing the brain organoid development in vitro. iPSC stands for induced pluripotent stem cells. NPC stands for neural progenitor cell.

FIG. 17 is a graph showing the replicability of brain organoid development from two independent experiments.

FIGS. 18A and 18B are tables showing the change in the expression level of certain genes in APP gene duplication organoid.

FIG. 19 is human genetic and postmortem brain analysis published data that independently corroborate biomarkers predicted from the Alzheimer's disease neural organoid derived data, including novel changes in microglial functions increasing susceptibility to infectious agents in Alzheimer's disease.

### DETAILED DESCRIPTION

Unless defined otherwise, all technical and scientific terms used herein have the same meaning as is commonly understood by one of ordinary skill in the art. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). These references are intended to be exemplary and illustrative and not limiting as to the source of information known to the worker of ordinary skill in this art. As used herein, the following terms have the meanings ascribed to them below, unless specified otherwise.

It is noted here that as used in this specification and the appended claims, the singular forms “a,” “an,” and “the” also include plural reference, unless the context clarity dictates otherwise.

The term “about” or “approximately” means within 25%, such as within 20% (or 5% or less) of a given value or range.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention, it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

A “neural organoid” means a non-naturally occurring three-dimensional organized cell mass that is cultured in vitro from a human induced pluripotent stem cell and develops similarly to the human nervous system in terms of neural marker expression and structure. Further, a neural organoid has two or more regions. The first region expresses cortical or retinal marker or markers. The remaining regions each express markers of the brain stem, cerebellum, and/or spinal cord.

Neural markers are any protein or polynucleotide expressed consistent with a cell lineage. By “neural marker”, it is meant any protein or polynucleotide, the expression of which is associated with a neural cell fate. Exemplary neural markers include markers associated with the hindbrain, midbrain, forebrain, or spinal cord. One skilled in the art will understand that neural markers are representative of the cerebrum, cerebellum and brainstem regions. Exemplary brain structures that express neural markers include the cortex, hypothalamus, thalamus, retina, medulla, pons, and lateral ventricles. Further, one skilled in the art will recognize that within the brain regions and structures, granular neurons, dopaminergic neurons, GABAergic neurons, cholinergic neurons, glutamatergic neurons, serotonergic neurons, dendrites, axons, neurons, neuronal, cilia, purkinje fibers, pyramidal cells, spindle cells, express neuronal markers. One skilled in the art will recognize that this list is not all encompassing and that neural markers are found throughout the central nervous system including other brain regions, structures, and cell types.

Exemplary cerebellar markers include but are not limited to ATOH1, PAX6, SOX2, LHX2, and GRID2. Exemplary markers of dopaminergic neurons include but are not limited to tyrosine hydroxylase, vesicular monoamine transporter 2 (VMAT2), dopamine active transporter (DAT) and Dopamine receptor D2 (D2R). Exemplary cortical markers include, but are not limited to, doublecortin, NeuN, FOXP2, CNTN4, and TBR1. Exemplary retinal markers include but are not limited to retina specific Guanylate Cyclases (GUY2D, GUY2F), Retina and Anterior Neural Fold Homeobox (RAX), and retina specific Amine Oxidase, Copper Containing 2 (RAX). Exemplary granular neuron markers include, but are not limited to SOX2, NeuroD1, DCX, EMX2, FOXG1I, and PROX1. Exemplary brain stem markers include, but are not limited to FGF8, INSM1, GATA2, ASCL I, GATA3. Exemplary spinal cord markers include, but are not limited to homeobox genes including but not limited to HOXA1, HOXA2, HOXA3, HOXB4, HOXAS, HOXCS, or HOXDI3. Exemplary GABAergic markers include, but are not limited to NKCCI or KCC2. Exemplary astrocytic markers include, but are not limited to GFAP. Exemplary oliogodendrocytic markers include, but are not limited to OLIG2 or MBP. Exemplary microglia markers include, but are not limited to AIF1 or CD4. In one embodiment, the measured biomarkers listed above have at least 70% homology to the sequences in the Appendix. One skilled in the art will understand that the list is exemplary and that additional biomarkers exist.

Diagnostic or informative alteration or change in a biomarker is meant as an increase or decrease in expression level or activity of a gene or gene product as detected by conventional methods known in the art such as those described herein. As used herein, such an alteration can include a 10% change in expression levels, a 25% change, a 40% change, or even a 50% or greater change in expression levels.

A mutation is meant to include a change in one or more nucleotides in a nucleotide sequence, particularly one that changes an amino acid residue in the gene product. The change may or may not have an impact (negative or positive) on activity of the gene.

Neural organoids are generated in vitro from patient Neural organoids are generated in vitro from patient tissue samples. Neural organoids were previously disclosed in WO2017123791A1 (https://patents.google.com/patent/WO2017123791A1/en), incorporated herein, in its entirety. A variety of tissues can be used including skin cells, hematopoietic cells, or peripheral blood mononuclear cells (PBMCs) or in vivo stem cells directly. One of skill in the art will further recognize that other tissue samples can be used to generate neural organoids. Use of neural organoids permits study of neural development in vitro. In one embodiment skin cells are collected in a petri dish and induced to an embryonic-like pluripotent stem cell (iPSC) that have high levels of developmental plasticity. iPSCs are grown into neural organoids in said culture under appropriate conditions as set forth herein and the resulting neural organoids closely resemble developmental patterns similar to human brain. In particular, neural organoids develop anatomical features of the retina, forebrain, midbrain, hindbrain, and spinal cord. Importantly, neural organoids express >98% of the about 15,000 transcripts found in the adult human brain. iPSCs can be derived from the skin or blood cells of humans identified with substantial changes in gene expression of the genes listed in Tables 1, 4, or 5 with corresponding sequences found in publicly available databases such as Genecards, Genbank, and Pubmed among others.

The neural organoid model and methods of diagnosing drug abuse susceptibility are advantageous over current tumor and cancer susceptibility models. Importantly, the neural organoid and associated methods herein allow for the study of drug abuse susceptibility in a native neural microenvironment with all the proper niche factors. The organoid drug abuse susceptibility model is human and native and this is advantageous for the epithelial-to-mesenchymal transition for identifying patterns drug abuse susceptibility progression in humans In one In one embodiment, the about 12-week old iPSC-derived human neural organoid has ventricles and other anatomical features characteristic of a 35-40 day old neonate. In an additional embodiment the about 12 week old neural organoid expresses beta 3-tubulin, a marker of axons as well as somato-dendritic Puncta staining for MAP2, consistent with dendrites. In yet another embodiment, at about 12 weeks the neural organoid displays laminar organization of cortical structures. Cells within the laminar structure stain positive for doublecortin (cortical neuron cytosol), Beta3 tubulin (axons) and nuclear staining. The neural organoid, by 12 weeks, also displays dopaminergic neurons and astrocytes. Accordingly, as noted, neural organoids permit study of human neural development in vitro.

Further, the neural organoid offers the advantages of replicability, reliability and robustness, as shown herein using replicate neural organoids from the same source of iPSCs. Importantly, an advantage of the methods using the neural organoid as disclosed herein is that they can be used to detect drug abuse susceptibility using an autologous sample from the patient. derived from a stem cell origin.

The neural organoid platform disclosed herein offers several advantages over pre-existing platforms. The platform is a single ‘multipurpose’ human pre-clinical medical device that can be used to identify the risk of drug abuse onset. The neural organoid is replicable, reproducible, robust and scalable. Importantly, the model is well suited for medical diagnostic & drug discovery. The platform is advantageous as it is a highly advanced, human brain model synthetically engineered in the dish. This allows for a wide range of—omic analysis on the neural organoid, including transcriptomic, proteomic, and metabolomics analyses. The neural organoid model disclosed herein contains all brain regions including the Retina, Cortex, Midbrain, Brain Stem, and Spinal Cord. The model expresses ˜15,000 genes that show >98% match to those of the adult human brain including all major central nervous cell types including neurons, astrocytes, oligodendrocytes, and microglia. Thus, in addition to efficacy in studying susceptibility to drug abuse the neural organoid model disclosed herein can be used to study morbidity associated with degenerative brain conditions.

In contrast, currently available organoid models such as spheroids, neurons-in a-dish, cortical, and cerebral organoids are less well developed human surrogate models with limited or little pre-clinical applicability. Importantly, these currently available models: (1) lack well known network connectivity across different brain regions; (2) lack reproducible results; (3) lack complexity of types including the microglia involved in early and known immune responses that are critical in many brain disorders including Alzheimer's (See Tanzi et al., 2017, Kriegstein 2017;and https://www.nasw.org/article/new-mini-brain-could-complement-replace-animal-models); and (4) fail to demonstrate comprehensive output data for any mental illness. Compared to the disclosed neural organoid, the lack of comprehensive output data is a critical missing factor in current models as without feedback-feedforward brain circuits formed early in development among the different brain regions and types including the microglia, it is not possible to fully replicate disorders.

### Developmental Transcriptomics

A “transcriptome” is a collection of all RNA, including messenger RNA (mRNA), long non-coding RNAs (IncRNA), microRNAs (miRNA) and, small nucleolar RNA snoRNA), other regulatory polynucleotides, and regulatory RNA (IncRNA, miRNA) molecules expressed from the genome of an organism through transcription therefrom. Thus, transcriptomics is the study of the mRNA transcripts produced by the genome at a given time in any particular cell or tissue of the organism. Transcriptomics employs high-throughput techniques to analyze genome expression changes associated with development or disease. In certain embodiments, transcriptomic studies can be used to compare normal, healthy tissues and diseased tissue gene expression. In further embodiments, mutated genes or variants associated with disease or the environment can be identified.

Consistent with this, the aim of developmental transcriptomics is identifying genes associated with, or significant in, organismal development and disease and dysfunctions associated with development. During development, genes undergo up- and down-regulation as the organism develops. Thus, transcriptomics provides insight into cellular processes, and the biology of the organism.

Generally, in one embodiment RNA is sampled from the neural organoid described herein within at about one week, about four weeks, or about twelve weeks of development; most particularly RNA from all three time periods are sampled. However, RNA from the neural organoid can be harvested at minutes, hours, days, or weeks after reprogramming. For instance, RNA can be harvested at about 10 minutes, 20 minutes, 30 minutes, 40 minutes, 50 minutes, and 60 minutes. In a further embodiment the RNA can be harvested 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours. In a further embodiment the RNA can be harvested at 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, or 1 week, 2 weeks, 3 weeks, 4 weeks, 5 weeks, 6 weeks, 7 weeks, 8 weeks, 9 weeks 10 weeks, 11 weeks, 12 weeks or more in culture. After enriching for RNA sequences, an expressed sequence tag (EST) library is generated and quantitated using the AmpliSeg™ technique from ThermoFisher. Exemplars of alternate technologies include RNASeq and chip based hybridization methods. Transcript abundance in such experiments is compared in control neural organoids from healthy individuals vs. neural organoids generated from individuals with disease and the fold change in gene expression calculated and reported.

Furthermore, in one embodiment RNA from neural organoids for drug abuse susceptibility, are converted to DNA libraries and then the representative DNA libraries are sequenced using exon-specific primers for 20,814 genes using the AmpliSeg™ technique available commercially from ThermoFisher. Reads in cpm <1 are considered background noise. All cpm data are normalized data and the reads are a direct representation of the abundance of the RNA for each gene.

Briefly, in one embodiment, the array consists of one or a plurality of genes used to predict risk of drug abuse susceptibility. In an alternative embodiment, reads contain a plurality of genes that are used to treat drug abuse and drug abuse susceptibility in a human, using patient-specific pharmacotherapy known to be associated with drug abuse. In one aspect, the gene libraries can be comprised of drug abuse specific genes as provided in Table 1. Exemplarily, changes in expression or mutation of drug abuse-specific genes are detected using such sequencing, and differential gene expression detected thereby, qualitatively by detecting a pattern of gene expression or quantitatively by detecting the amount or extent of expression of one or a plurality of disease-specific genes or mutations thereof. Results of said assays using the AmpliSeg™ techniques are used to identify genes that can predict or identify drug abuse susceptibility or onset and can be targets of therapeutic intervention. In further embodiments, hybridization assays can be used, including but not limited to sandwich hybridization assays, competitive hybridization assays, hybridization-ligation assays, dual ligation hybridization assays, or nuclease assays.

### Neural Organoids and Pharmaceutical Testing

Neural organoids are useful for pharmaceutical testing. Currently, drug screening studies including toxicity, safety and or pharmaceutical efficacy, are performed using a combination of in vitro work, rodent/primate studies and computer modeling. Collectively, these studies seek to model human responses, in particular physiological responses of the central nervous system.

Human neural organoids are advantageous over current pharmaceutical testing methods for several reasons. First neural organoids are derived from healthy and diseased patients, mitigating the need to conduct expensive clinical trials. Second, rodent models of human disease are unable to mimic physiological nuances unique to human growth and development. Third, use of primates creates ethical concerns. Finally, current methods are indirect indices of drug safety. Alternatively, neural organoids offer an inexpensive, easily accessible model of human brain development. This model permits direct, and thus more thorough, understanding of the safety, efficacy, and toxicity of pharmaceutical compounds.

Starting material for neural organoids is easily obtained from healthy and diseased patients. Further, because human organoids are easily grown they can be produced en mass. This permits efficient screening of pharmaceutical compounds.

Neural organoids are advantageous for identifying biomarkers of a disease or a condition, the method comprising a) obtaining a biological sample from a human patient; and b) detecting whether at least one biomarker is present in the biological sample by contacting the biological sample with an array comprising binding molecules specific for the biomarkers and detecting binding between the at least one biomarker and the specific binding molecules. In further embodiments, the biomarker serves as a gene therapy target.

Consistent with this need, neural organoids hold significant promise for studying drug abuse susceptibility as well as addiction and addictive behavior. Neural organoids are developed from cell lineages that have been first been induced to become pluripotent stem cells. Thus, the neural organoid is patient specific. Importantly, such models provide a method for studying neurological diseases and disorders that overcome previous limitations. Accordingly, the model can be used to develop patient-specific reagents, therapeutic modalities, and methods based on predictive biomarkers for diagnosing and/or treating current and future risk of drug abuse as well as therapeutic countermeasures.

### Developmental Transcriptomics and Predictive Medicine

Changes in gene expression of specific genes when compared to those from non-diseased samples by >1.4 fold identify candidate genes correlating with a disease. Further searches of these genes in data base searches (e.g. Genecard, Malacard, Pubmed; Human Protein Atlas (https://www.proteinatlas.org/ENSG00000115091-ACTR3/pathology) identify known diseases correlated previously with the disease state. In one embodiment AmpliSeg™ quantification of fold expression change allows for determination of fold change from control.

### Drug Abuse Susceptibility

Drug abuse and addiction is a chronic disease with nicotine addiction widely known as a gateway drug. Alcohol is the most widely abused drug in the United States while over two million people have an opioid addiction disorder (Addiction Center Statistics). Drug abuse affects the daily lives of addicts making daily activities difficult and is associated with a range of social-economic related pressures and outcomes. For instance, drug abuse is associated with drunken driving, inability to maintain employment, crime, financial stress, and violence.

Unfortunately, one of every eight adults suffers from both alcohol and drug disorders while 8.5 million American adults suffer from a mental health disorder in addition to addiction disorder. Despite this, less than 20% of addicts seek treatment, despite addiction being a manageable disease with the possibility of recovery. American Addiction Centers. Alcohol and Drug Abuse Statistics, accessed Aug. 12, 2019 (https://americanaddictioncenters.org/rehab-guide/addiction-statistics.

Importantly, genetic predisposition accounts for 40%-60% of the total risk of addiction. The neural organoid model described herein is advantageous as it can be used to detect the presence of biomarkers associated with addiction. This overcomes the limitations of treatment models that are implemented only after the participation in addictive behaviors or drug abuse onset. Thus, the model is advantageous in that it can be used to predict risk of addiction in an individual based on genetics prior to drug abuse.

Drug abuse and addiction encompasses a wide range of compounds including, but not limited to, nicotine, alcohol, opioids, heroin, cocaine, and methamphetamine. In one embodiment a method for predicting risk drug abuse susceptibility in a human, the method comprising: procuring one or a plurality of cell samples from the human, comprising one or a plurality of cell types; reprogramming the one or the plurality of cell samples to produce one or a plurality of induced pluripotent stem cell samples; treating the one or the plurality of induced pluripotent stem cell samples to obtain a neural organoid; collecting a biological sample from the neural organoid; measuring biomarkers in the neural organoid sample; and detecting measured biomarkers from the neural organoid sample that are differentially expressed in drug abuse and drug abuse susceptibility. .

Our preliminary data indicate that a subset of genes (i.e. Nurr 1, Lmx1a, Lmx1b, Neurog2, OTX2, Nolz1, NDNF) that are upregulated by nicotine support that our human neural organoids express genes for the dopamine cell fate determinants (Blaess and Ang; 2015) (Shh, Lmx1, En1 and 2, Fgf 8, Nurr1, and Pitx3), dopamine biosynthesis (TH), dopamine receptors (DR1-4), and dopamine transporters (SL18A2 and 2) typical of the midbrain after ˜12 weeks in culture. This success has allowed us to explore whether the expression of these genes reflect proper midbrain development in the human stem cell brain model.

### Opioid Pain Medication and Opioid Addiction

Opioid addiction is a complex condition characterized by an ongoing and compulsive need to obtain and use opioid drugs. The compounds have a high addictive potential in certain individuals and those addicted to opioids often have severe physical, mental, and economic consequences associated with opioid addiction. NIH Genetics Home Reference https://ghr.nlm.nih.gov/condition/opioid-addiction#inheritance.

Common opioids include oxycodone, fentanyl, buprenorphine, methadone, oxymorphone, hydrocodone, codeine, and morphine as well as non-prescription drugs such as heroin. Physiologically the drugs change the chemistry of the brain resulting in drug tolerance, and eventually dependence that can evolve to an addiction to opioids.

Opioid addiction results from a combination of genetic, environmental, and lifestyle factors. Id. Genetically, many genes identified in opioid addiction are involved in the endogenous opioid system. Opioid receptors are often located, for example, in the outer membrane of neurons with the mu (μ) opioid receptor, produced from the OPRM1 gene, being the primary receptor for most opioid drugs. Mutations in the OPRM1 gene and μ opioid receptor appear to play a key role in the physiological response to opioids. Additional genes, involved in nervous system function and neurotransmitter action are also implicated in opioid addiction. These and other novel genes are described herein providing support to the neural organoid model for predicting risk of drug abuse susceptibility including abuse of opioids. Id.

Recent meta-analysis of more than 175,000 individuals provides strong support to reaffirm that tobacco addiction is a gateway drug correlated with opioid use and opioid use disorders. Rajabi et al. 2019 Addict Behay. 95: 225-235, 2019.

### Non-Addictive Pain Medication

The opioid addiction crisis could be mitigated in-part through the availability of non-addictive pain medication. However, there is currently a scarcity of non-addictive pain killers that provide adequate pain management. Moreover, development of novel compounds is expensive and requires medicinal chemistry, rodent, and human studies.

The disclosed neural organoid provides a novel model for developing new, non-addictive pain medicine. Importantly, because the neural organoid is unique to an individual and has all brain regions it allows for the study of the biological, physiological, and pharmacological impact of new non-addictive drugs without the use of rodent and human studies. Furthermore, because the starting material is a fibroblast or similar cell the model is cost effective and personalized compared to traditional methods of drug development. An advantage of the current model in identifying non-addictive pain medication is that the neural organoid model reflects the dynamic microglia expression pattern during phases of development. Microglia are known to express distinct sets of genes in the early (E10.4-14), pre-microglia [E14-postnatal day (P) 9], and adult microglia (P28 and on) phases of development. Lenz and Nelson, 2018 Frontiers in Immunology 9: Article 608. The neural organoid used with the methods herein reflects this dynamic microglia gene expression. Further, expression of the NR4A1 biomarker, a key biomarker for use in developing non-addictive is expressed in the neural organoid. This and other features of the neural organoid provide an ideal model for use in developing new, non-addictive pain drugs as well as identifying the risk of drug abuse susceptibility.

In a particular embodiment, at least one cell sample, such as a fibroblast, is reprogrammed to become a pluripotent stem cell. In one embodiment, the fibroblast is a skin cell that is induced to become a neural organoid after being reprogrammed to become a pluripotent stem cell. In a particular embodiment, the neural organoid is harvested at about 10 minutes to 12 weeks post-inducement. At each time point, the RNA is isolated and the gene biomarkers measured. The measured biomarkers comprise nucleic acids or proteins. In a particular embodiment, the measured biomarker is a nucleic acid encoding human genes as listed in Table 1 or variants thereof.

Drug abuse and drug abuse susceptibility are associated with changes in gene expression. The terms biomarker means any nucleic acid sequence encoding the respective polypeptide having at least 70% homology to the sequences in Table 1.

Although the expression of multiple genes is altered in drug abuse and drug abuse susceptibility, in one-embodiment lead candidate genes can be used to identify therapeutic targets as listed in Table 1 or Table 4. In a further embodiment, the measured biomarkers mean any nucleic acid sequence encoding the respective polypeptide having at least 70% homology to the gene accession numbers listed in Table 1.

In another aspect, the genes listed in Tables 1, 4, and 5 can be used to predict the risk of drug abuse later in life. In a further embodiment, the measured biomarkers mean any nucleic acid sequence encoding the respective polypeptide having at least 70% homology to the gene accession numbers listed in Table 1.

The skilled worker will recognized the exemplarily markers as set forth in Table 1 to be human-specific maker proteins as identified, inter alia, in genetic information repositories such as GenBank, GeneCards, and Pubmed, among others. In addition, drug abuse susceptibility can be predicted using detection of a combination of drug abuse susceptibility biomarkers or variants thereof as provided in Table 1.

One skilled in the art will also recognize that the lead genes set forth herein are not exhaustive and additional genes and gene combinations can also be used to predict the risk of drug abuse susceptibility.

In one aspect, at least one cell sample reprogrammed to the induced pluripotent stem cell is a fibroblast derived from skin or blood cells from humans. In another aspect the fibroblast-derived skin or blood cells from humans is identified with the genes identified in Table 1 or Table 4 with corresponding sequences found in publicly available databases such as Genecards, Genbank, and Pubmed among others. In yet another aspect, the measured biomarkers comprise nucleic acids, proteins, or their metabolites. In another aspect, the measured biomarkers comprise one or a plurality of biomarkers identified in Table 1, or Table 4 or variants thereof with corresponding sequences and variants of sequences found in publicly available databases such as Genecards, Genbank, and Pubmed among others. In yet another aspect, a combination of multi-organ tumor & cancer susceptibility biomarkers are detected, the combination comprising a nucleic acid encoding human NURR1, Lmx1a, Lmx1b, Neurog2, OTX2, Nolz1, and NDNF, and associated variants; and one or a plurality of biomarkers comprising a nucleic acid encoding human genes identified in Table 1 (and dose-responses to nicotine which identify nicotinic receptor subtype specificity (alpha 7 as an exemplar).

In one aspect of the disclosure, the biomarkers for drug abuse susceptibility include human nucleic acids, proteins, or their metabolites as listed in Table 1.

In still another aspect, the neural organoid biological sample is collected after about one hour up to about 12 weeks post inducement. In another aspect, the neural organoid sample is procured from structures of the neural organoid that mimic structures developed in utero at about 5 weeks. In yet another aspect the neural organoid at about twelve weeks post-inducement comprises structures and cell types of retina, cortex, midbrain, hindbrain, brain stem, or spinal cord. In a one aspect, the neural organoid contains microglia, and one or a plurality of drug abuse susceptibility biomarkers as identified in Table 1.

The detection of novel biomarkers, as presented in Table 1 and/or Tables 4 and 5 can be used to identify individuals who should be provided prophylactic treatment for drug abuse susceptibility. In one aspect, early diagnosis can be used in a personalized medicine approach to identify new patient specific pharmacotherapies for drug abuse susceptibility based on biomarker data. In a further aspect, the neural organoid model can be used to test the effectiveness of currently utilized drug abuse and drug abuse susceptibility therapies. In one aspect, the neural organoid could be used to identify the risk and/or onset of drug abuse susceptibility and additionally, provide patient-specific insights into the efficacy of using known pharmacological agents to treat drug abuse or drug abuse susceptibility. This allows medical professionals to identify and determine the most effective treatment for a drug abuse susceptible patient, before symptoms arise. Furthermore, one skilled in the art will recognize that the effectiveness of additional FDA-approved, as well as novel drugs under development could be tested using the methods disclose herein. In a further aspect, the method allows for development and testing of non-individualized, global treatment strategies for mitigating the effects and onset of drug abuse susceptibility.

In a second embodiment, the disclosure provides diagnostic methods for predicting risk for drug abuse susceptibility in a human, comprising one or a plurality subset of the biomarkers as identified in Table 1 or Table 4. In yet another aspect, the subset of measured biomarkers comprise nucleic acids, proteins, or their metabolites as identified in Table 1 or Table 4 with corresponding sequences found in publicly available databases such as Genecards, Genbank, and Pubmed among others.

In a third embodiment are methods of pharmaceutical testing for drug abuse susceptibility drug screening, toxicity, safety, and/or pharmaceutical efficacy studies using patient-specific neural organoids.

In a fourth embodiment, methods are provided for detecting at least one biomarker of drug abuse susceptibility , the method comprising, obtaining a biological sample from a human patient; and contacting the biological sample with an array comprising specific-binding molecules for the at least one biomarker and detecting binding between the at least one biomarker and the specific binding molecules. In one aspect, the biomarker detected is a gene therapy target.

In a fifth embodiment the disclosure provides a kit comprising an array containing sequences of biomarkers from Table 1 for use in a human patient. In one aspect, the kit further contains reagents for RNA isolation and biomarkers for drug abuse susceptibility. In a further aspect, the kit further advantageously comprises a container and a label or instructions for collection of a sample from a human, isolation of cells, inducement of cells to become pluripotent stem cells, growth of patient-specific neural organoids, isolation of RNA, execution of the array and calculation of gene expression change and prediction of concurrent or future disease risk. In one aspect, biomarkers can comprise any markers in Table 1 or variants thereof.

In a sixth embodiment, the disclosure provides a method for detecting one or a plurality of biomarkers from different human chromosomes associated with drug abuse susceptibility using data analytics that obviates the need for whole genome sequence analysis of patient genomes. In one aspect, the methods are used to determine gene expression level changes that are used to identify clinically relevant symptoms and treatments, and addiction severity. In yet another aspect, the neural organoids are used to identify novel biomarkers that serve as data input for development of algorithm techniques as predictive analytics. In a further aspect, the algorithmic techniques include artificial intelligence, machine and deep learning as predictive analytics tools for identifying biomarkers for diagnostic, therapeutic target and drug development process for disease. Gene expression measured in drug abuse susceptibility can encode a variant of a biomarker alterations encoding a nucleic acid variant associated with drug abuse susceptibility. In one embodiment, the nucleic acid encoding the variant is comprised of one or more missense variants, missense changes, or enriched gene pathways with common or rare variants.

In an alternative embodiment the method for predicting a risk for developing drug abuse susceptibility in a human, comprising: collecting a biological sample; measuring biomarkers in the biological sample; and detecting measured biomarkers from the sample that are differentially expressed in humans with a susceptibility to drug abuse wherein the measured biomarkers comprise those biomarkers encoding human biomarkers or variants listed as listed in Table 1. In one aspect, a plurality of biomarkers comprising a diagnostic panel for predicting a risk for drug abuse susceptibility in humans, comprising biomarkers listed in Table 1, or variants thereof. In one aspect of the embodiment a subset of marker can be used, wherein the subset comprises a plurality of biomarkers from 2 to 200, or 2-150, 2-100, 2-50, 2-25, 2-20, 2-15, 2-10, or 2-5 genes.

In yet another embodiment the measured biomarker is a nucleic acid panel for predicting drug abuse susceptibility in humans. The genes encoding the biomarkers listed in Table 1 or variants thereof. Said panel can be provided according to the invention as an array of diagnostically relevant portions of one or a plurality of these genes, wherein the array can comprise any method for immobilizing, permanently or transiently, said diagnostically relevant portions of said one or a plurality of these genes, sufficient for the array to be interrogated and changes in gene expression detected and, if desired, quantified. In alternative embodiments, the array comprises specific binding compounds for binding to the protein products of the one or a plurality of these genes. In yet further alternative embodiments, said specific binding compounds can bind to metabolic products of said protein products of the one or a plurality of these genes. In one aspect drug, abuse susceptibility is predicted or detected by expression of one or a plurality of biomarkers as identified in Table 5.

Another embodiment of the invention disclosed herein uses the neural organoids derived from the human patient in the non-diagnostic realm. The neural organoids express markers characteristic of a large variety of neurons and include markers for astrocytic, oligodendritic, microglial, and vascular cells. The neural organoids form all the major regions of the brain including the retina, cortex, midbrain, brain stem, and the spinal cord in a single brain structure expressing greater than 98% of the genes known to be expressed in the human brain. Such characteristics enable the neural organoid to be used as a biological platform/device for drug screening, toxicity, safety, and/or pharmaceutical efficacy studies understood by those having skill in the art. Additionally, since the neural organoid is patient specific, pharmaceutical testing using the neural organoid allows for patient specific pharmacotherapy.

In yet another embodiment the disclosure provides methods for predicting a risk for developing drug abuse in a human, the method comprising procuring one or a plurality of cell samples from a human, comprising one or a plurality of cell types; reprogramming the one or the plurality of cell samples to produce one or a plurality of induced pluripotent stem cell samples; treating the one or the plurality of induced pluripotent stem cell samples to obtain one or more patient specific neural organoids; collecting a biological sample from the patient specific neural organoid; measuring biomarkers in the neural organoid sample; and detecting measured biomarkers from the neural organoid sample that are differentially expressed in humans that abuse drugs. In one aspect, the cell sample reprogrammed to the induced pluripotent stem cell is a fibroblast. In certain aspects, the measured drug abuse susceptibility biomarkers comprise nucleic acids, proteins, or their metabolites. In further aspects, the measured biomarker is a nucleic acid encoding human NURR1, Lmx1a, Lmx1b, Neurog2, OTX2, Nolz1, and NDNF and associated variants. In further aspects, the measured biomarkers comprise one or a plurality of genes as identified in Tables 1, 4 or 5. In additional aspects, the neural organoid sample is procured from minutes to hours up to 15 weeks post inducement, wherein the biomarkers to be tested are one or a plurality of biomarkers in Tables 1, 4, or 5.

These and other data findings, features, and advantages of the present disclosure will be more fully understood from the following detailed description taken together with the accompanying claims. It is noted that the scope of the claims is defined by the recitations therein and not by the specific discussion of features and advantages set forth in the present description.

### EXAMPLES

The Examples that follow are illustrative of specific embodiments of the invention, and the use thereof. It is set forth for explanatory purposes only and is not taken as limiting the invention. In particular, the example demonstrates the effectiveness of neural organoids in predicting future disease risk.

### Materials and Methods

The neural organoids described above were developed using the following materials and methods.

### Summary of Methods:

Neural Organoids derived from induced pluripotent stem cells derived from adult skin cells of patients were grown in vitro for 4 weeks as previous described in our PCT Application (PCT/US2017/013231). Transcriptomic data from these neural organoids were obtained. Differences in expression of 20,814 genes expressed in the human genome were determined between these neural organoids and those from neural organoids from a normal individual human. Detailed data analysis using Gene Card and Pubmed data bases were performed. Genes that were expressed at greater than 1.4-fold following exposure for 4 weeks to 0 (control) versus nicotine at doses of 50 nanomolar, 500 nanomolar, and 1 micromolar in the growth media were identified. These are found to be highly significant because a vast majority were correlated with genes previously associated with a multitude of neurodevelopmental and neurodegenerative diseases as well as those found to be dysregulated in post mortem patient brains. These genes responsive to nicotine comprise a suite of biomarkers for drug abuse susceptibility.

The invention advantageously provides many uses, including but not limited to a) early diagnosis of these diseases at birth from new born skin cells; b) Identification of biochemical pathways that increase environmental and nutritional deficiencies in new born infants; c) discovery of mechanisms of disease mechanisms; d) discovery of novel and early therapeutic targets for drug discovery using timed developmental profiles; e) testing of safety, efficacy and toxicity of drugs in these pre-clinical models.

Cells used in these methods include human iPSCs, feeder-dependent (System Bioscience. WT SC600A-W) and CF-1 mouse embryonic fibroblast feeder cells, gamma-irradiated (Applied StemCell, Inc #ASF-1217)

Growth media or DMEM media, used in the examples contained the supplements as provided in Table 2 (Growth Media and Supplements used in Examples).

TABLE 2
Growth Media and Supplements used in Examples

| Media/Supplement | Vendor/Catalog Number |
|---|---|
| DMEM non-essential amino acids | MEM-NEAA, Invitrogen #11140-050 |
| Phosphate Buffered Saline, sterile | Invitrogen #14040-091 |
| Phosphate Buffered Saline, | Invitrogen #14190-094 |
| Ca++ and Mg++ free | |
| Gentamicin Reagent Solution | Invitrogen #15750-060 |
| Antibiotic-Antimycotic | Invitrogen #15240-062 |
| 2-mercaptoethanol | EmbryoMAX, |
| | EMBMillipore#ES-007-E |
| Basic fibroblast growth factor | FGF, PeproTech #051408-1 |
| Heparin | Sigma, #H3149-25KU |
| Insulin solution | Sigma #I9278-5ml |
| Dimethyl sulfoxide | Millipore #D9170-5VL |
| ROCK Inhibitor Y27632 | Millipore#SCM075 |
| Gelatin solution, Type B | Sigma #GI 393-100 ml |
| Matrigel Matrix NOT Growth | BD Bioscience #354234 |
| Factor Reduced Matrigel | |
| Accutase | Sigma #A6964 |
| Hydrogen Peroxide | Fisher #H325-500 |
| Ethanol | |
| Sterile H20 | |

One skilled in the art will recognize that additional formulations of media and supplements can be used to culture, induce and maintain pluripotent stem cells and neural organoids.

Experimental protocols required the use of multiple media compositions including MEF Media, IPSO Media, EB Media, Neural Induction Media, and Differentiation Medias 1, 2, and 3.

Mouse embryonic fibroblast (MEF) was used in cell culture experiments. MEF Media comprised DMEM media supplemented with 10% Feta Bovine Serum, 100 units/ml penicillin, 100 microgram/ml streptomycin, and 0.25 microgram/ml Fungizone.

Induction media for pluripotent stem cells (IPSO Media) comprised DMEM/F12 media supplemented with 20% Knockout Replacement Serum, 3% Fetal Bovine Serum with 2 mM Glutamax, IX Minimal Essential Medium Nonessential Amino Acids, and 20 nanogram/ml basic Fibroblast Growth Factor

Embryoid Body (EB) Media comprised Dulbecco's Modified Eagle's Medium (DMEM) (DMEM)/Ham's F-12 media, supplemented with 20% Knockout Replacement Serum, 3% Fetal Bovine Serum containing 2 mM Glutamax, IX Minimal Essential Medium containing Nonessential Amino Acids, 55microM beta-mercaptoethanol, and 4 ng/ml basic Fibroblast Growth Factor.

Neural Induction Media contained DMEM/F12 media supplemented with: a 1:50 dilution N2 Supplement, a 1:50 dilution GlutaMax, a 1:50 dilution MEM-NEAA, and 10 microgram/ml Heparin'

Three differentiation medias were used to produce and grow neural organoids. Differentiation Media 1 contained DMEM/F12 media and Neurobasal media in a 1:1 dilution. Each media is commercially available from Invitrogen. The base media was supplemented with a 1:200 dilution N2 supplement, a 1:100 dilution B27—vitamin A, 2.5 microgram/ml insulin, 55 microMolar beta-mercaptoethanol kept under nitrogen mask and frozen at −20° C., 100 units/ml penicillin, 100 microgram/ml streptomycin, and 0.25 microgram/ml Fungizone.

Differentiation Media 2 contained DMEM/F12 media and Neurobasal media in a 1:1 dilution supplemented with a 1:200 dilution N2 supplement, a 1:100 dilution B27 containing vitamin A, 2.5 microgram/ml Insulin, 55 umicroMolar beta-mercaptoethanol kept under nitrogen mask and frozen at −20° C., 100 units/ml penicillin, 100 microgram/ml streptomycin, and 0.25 microgram/ml Fungizone.

Differentiation Media 3 consisted of DMEM/F12 media: Neurobasal media in a 1:1 dilution supplemented with 1:200 dilution N2 supplement, a 1:100 dilution B27 containing vitamin A), 2.5 microgram/ml insulin, 55 microMolar beta-mercaptoethanol kept under nitrogen mask and frozen at −20° C., 100 units/ml penicillin, 100 microgram/ml streptomycin, 0.25 microgram/ml Fungizone, TSH, and Melatonin.

The equipment used in obtaining, culturing and inducing differentiation of pluripotent stem cells is provided in Table 3 (Equipment used in Experimental Procedures). One skilled in the art would recognize that the list is not at all exhaustive but merely exemplary.

TABLE 3
Equipment used in Experimental Procedures

| | |
|---|---|
| StemPro EZPassage | Invitrogen#23181-010 |
| Tissue Culture Flasks, 115 cm² reclosable | TPP #TP90652 |
| Tissue Culture Flask, 150 cm² reclosable | TPP#TP90552 |
| Lipidure coat plate, 96 wells, U-bottom | LCU96 |
| Lipidure coat MULTI dish, 24 well | 510101619 |
| Parafilm | Sigma #P7793 |
| Sterile Filtration Units for 150 ml/250 ml | Sigma #TPP99150/ |
| solutions | TPP99250 |
| Benchtop Tissue Culture Centrifuge | ThermoFisher |
| C0₂ incubator, maintained at 37° C. and 5% C0₂ | ThermoFisher |
| Bench top rotary shaker | ThermoFisher |
| Light Microscope | Nikon |
| Confocal Microscope | Nikon |

### Example 1

### Generation of Human Induced Pluripotent Stem Cell-Derived Neural Organoids

Human induced pluripotent stem cell-derived neural organoids were generated according to the following protocol, as set forth in International Application No. PCT/US2017/013231 incorporated herein by reference. Briefly, irradiated murine embryonic fibroblasts (MEF) were plated on a gelatin coated substrate in MEF media (Dulbecco's Modified Eagle Medium (DMEM) supplemented with 10% Feta Bovine Serum, 100 units/ml penicillin, 100 microgram/ml streptomycin, and 0.25 microgram/ml Fungizone) at a density of 2×⁵ cells per well. The seeded plate was incubated at 37° C. overnight.

After incubation, the MEFs were washed with pre-warmed sterile phosphate buffered saline (PBS). The MEF media was replaced with 1 mL per well of induced pluripotent stem cell (iPSC) media containing Rho-associated protein kinase (ROCK) inhibitor. A culture plate with iPSCs was incubated at 37° C. The iPSCs were fed every other day with fresh iPSC media containing ROCK inhibitor. The iPSC colonies were lifted, divided, and transferred to the culture wells containing the MEF cultures so that the iPSC and MEF cells were present therein at a 1:1 ratio. Embryoid bodies (EB) were then prepared. Briefly, a 100 mm culture dish was coated with 0.1% gelatin and the dish placed in a 37° C. incubator for 20 minutes, after which the gelatin-coated dish was allowed to air dry in a biological safety cabinet. The wells containing iPSCs and MEFs were washed with pre-warmed PBS lacking Ca²⁺/Mg²⁺. A pre-warmed cell detachment solution of proteolytic and collagenolytic enzymes (1 mL/well) was added to the iPSC./MEF cells. The culture dishes were incubated at 37° C. for 20 minutes until cells detached. Following detachment, pre-warmed iPSC media was added to each well and gentle agitation used to break up visible colonies. Cells and media were collected and additional pre-warmed media added, bringing the total volume to 15 mL. Cells were placed on a gelatin-coated culture plate at 37° C. and incubated for 60 minutes, thereby allowing MEFs to adhere to the coated surface. The iPSCs present in the cell suspension were then counted.

The suspension was then centrifuged at 300×g for 5 minutes at room temperature, the supernatant discarded, and cells re-suspended in EB media supplemented with ROCK inhibitor (50 uM final concentration) and 4 ng/ml basic Fibroblast Growth Factor to a volume of 9,000 cells/150 μL. EB media is a mixture of DMEM/Ham's F-12 media supplemented with 20% Knockout Replacement Serum, 3% Fetal Bovine Serum (2 mM Glutamax), 1× Minimal Essential Medium Nonessential Amino Acids, and 55 μM beta-mercaptoethanol. The suspended cells were plated (150 μL) in a LIPIDURE® low-attachment U-bottom 96-well plate and incubated at 37° C.

The plated cells were fed every other day during formation of the embryoid bodies by gently replacing three fourths of the embryoid body media without disturbing the embryoid bodies forming at the bottom of the well. Special care was taken in handling the embryoid bodies so as not to perturb the interactions among the iPSC cells within the EB through shear stress during pipetting. For the first four days of culture, the EB media was supplemented with 50 uM ROCK inhibitor and 4 ng/ml bFGF. During the remaining two to three days the embryoid bodies were cultured, no ROCK inhibitor or bFGF was added.

On the sixth or seventh day of culture, the embryoid bodies were removed from the LIPIDURE® 96 well plate and transferred to two 24-well plates containing 500 pL/well Neural Induction media, DMEM/F12 media supplemented with a 1:50 dilution N2 Supplement, a 1:50 dilution GlutaMax, a 1:50 dilution MEM-Non-Essential Amino Acids (NEAA), and 10 μg/ml Heparin. Two embryoid bodies were plated in each well and incubated at 37° C. The media was changed after two days of incubation. Embryoid bodies with a “halo” around their perimeter indicate neuroectodermal differentiation. Only embryoid bodies having a “halo” were selected for embedding in matrigel, remaining embryoid bodies were discarded.

Plastic paraffin film (PARAFILM) rectangles (having dimensions of 5 cm×7 cm) were sterilized with 3% hydrogen peroxide to create a series of dimples in the rectangles. This dimpling was achieved, in one method, by centering the rectangles onto an empty sterile 200 μL tip box press, and pressing the rectangles gently to dimple it with the impression of the holes in the box. The boxes were sprayed with ethanol and left to dry in the biological safety cabinet.

Frozen Matrigel matrix aliquots (500 μL) were thawed on ice until equilibrated at 4° C. A single embryoid body was transferred to each dimple of the film. A single 7 cm×5 cm rectangle holds approximately twenty (20) embryoid bodies. Twenty microliter (20 μL) aliquots of Matrigel were transferred onto the embryoid bodies after removing extra media from the embryoid body with a pipette. The Matrigel was incubated at 37° C. for 30 min until the Matrigel polymerized. The 20 μL droplet of viscous Matrigel was found to form an optimal three-dimensional environment that supported the proper growth of the neural organoid from embryoid bodies by sequestering the gradients of morphogens and growth factors secreted by cells within the embryoid bodies during early developmental process. However, the Matrigel environment permitted exchange of essential nutrients and gases. Gentle oscillation by hand twice a day for a few minutes within a tissue culture incubator (37° C./5%CO₂) further allowed optimal exchange of gases and nutrients to the embedded embryoid bodies.

Differentiation Media 1, a one-to-one mixture of DMEM/F12 and Neurobasal media supplemented with a 1:200 dilution N2 supplement, a 1:100 dilution B27—vitamin A, 2.5 μg/mL insulin, 55 microM beta-mercaptoethanol kept under nitrogen mask and frozen at −20° C., 100 units/mL penicillin, 100 μg/mL streptomycin, and 0.25 μg/mL Fungizone, was added to a 100 mm tissue culture dish. The film containing the embryoid bodies in Matrigel was inverted onto the 100 mm dish with differentiation media 1 and incubated at 37° C. for 16 hours. After incubation, the embryoid body/Matrigel droplets were transferred from the film to the culture dishes containing media. Static culture at 37° C. was continued for 4 days until stable neural organoids formed.

Organoids were gently transferred to culture flasks containing differentiation media 2, a one-to-one mixture of DMEM/F12 and Neurobasal media supplemented with a 1:200 dilution N2 supplement, a 1:100 dilution B27+vitamin A, 2.5 μg/mL insulin, 55 microM beta-mercaptoethanol kept under nitrogen mask and frozen at −20° C., 100 units/mL penicillin, 100 pg/mL streptomycin, and 0.25 μg/mL Fungizone. The flasks were placed on an orbital shaker rotating at 40 rpm within the 37° C./5% CO₂ incubator.

The media was changed in the flasks every 3-4 days to provide sufficient time for morphogen and growth factor gradients to act on targets within the recipient cells forming relevant structures of the brains. Great care was taken when changing media so as to avoid unnecessary perturbations to the morphogen/secreted growth factor gradients developed in the outer most periphery of the organoids as the structures grew into larger organoids.

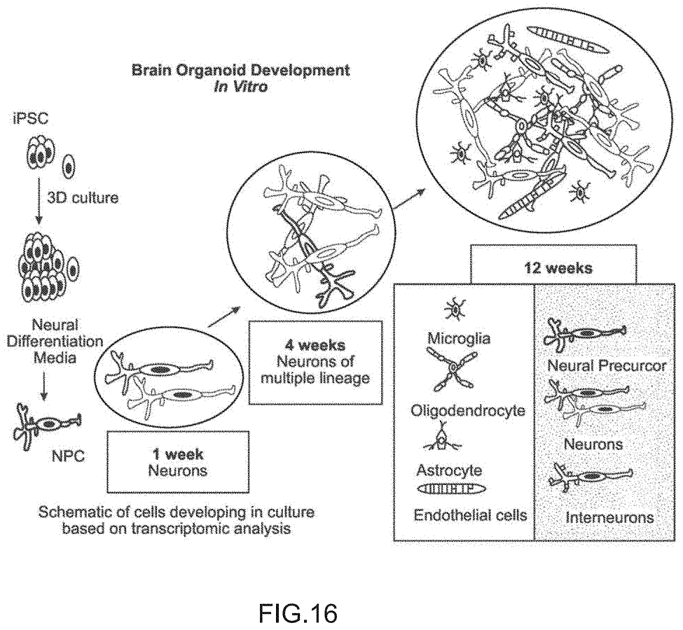

FIG. 16 illustrates neural organoid development in vitro. Based on transcriptomic analysis, iPSC cells form a body of cells after 3D culture, which become neural progenitor cells (NPC) after neural differentiation media treatment. Neurons were observed in the cell culture after about one week. After about four (4) weeks or before, neurons of multiple lineage appeared. At about twelve (12) weeks or before, the organoid developed to a stage having different types of cells, including microglia, oligodendrocyte, astrocyte, neural precursor, neurons, and interneurons.

### Example 2

### Human Induced Pluripotent Stem Cell-Derived Neural Organoids Express Characteristics of Human Brain Development

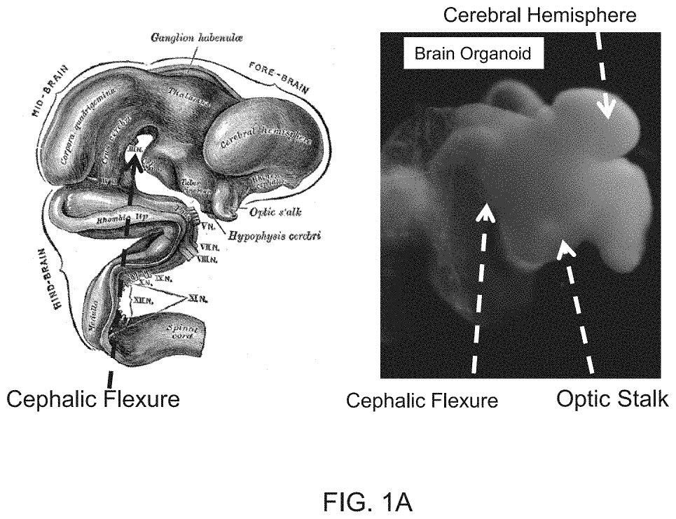

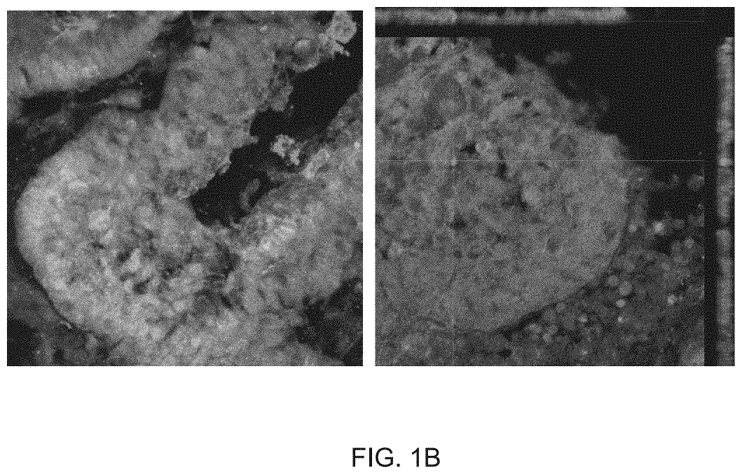

After approximately 12 weeks of in vitro culture, transcriptomic and immunohistochemical analysis indicated that organoids were generated according to the methods delineated in Example 1. Specifically, the organoids contained cells expressing markers characteristic of neurons, astrocytes, oligodendrocytes, microglia, and vasculature (FIGS. 1-14) and all major brain structures of neuroectodermal derivation. Morphologically identified by bright field imaging, the organoids included readily identifiable neural structures including cerebral cortex, cephalic flexure, and optic stalk (compare, Grey's Anatomy Textbook). The gene expression pattern in the neural organoid was >98% concordant with those of the adult human brain reference (Clontech, #636530). The organoids also expressed genes in a developmentally organized manner described previously (e.g. for the midbrain mesencephalic dopaminergic neurons; Blaese et al., Genetic control of midbrain dopaminergic neuron development. Rev Dev Biol. 4(2): 113-34, 2015). The structures also stained positive for multiple neural specific markers (dendrites, axons, nuclei), cortical neurons (Doublecortin), midbrain dopamine neurons (Tyrosine Hydroxylase), and astrocytes (GFAP) as shown by immunohistology).

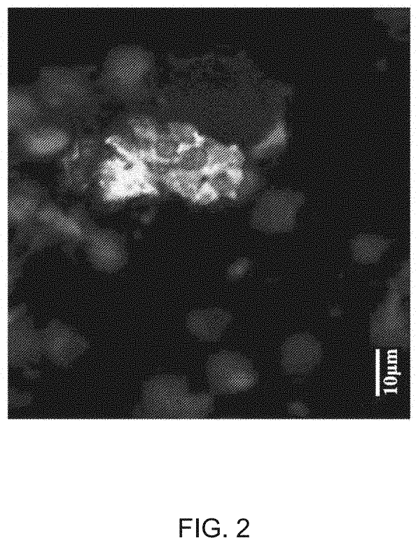

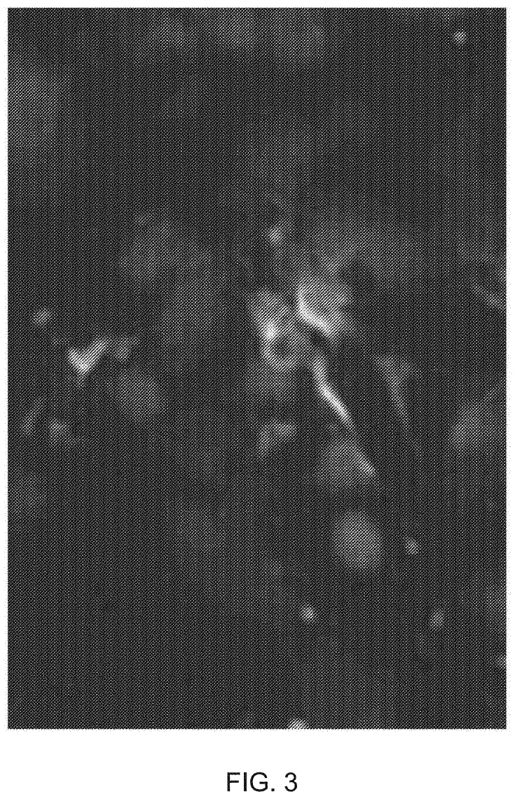

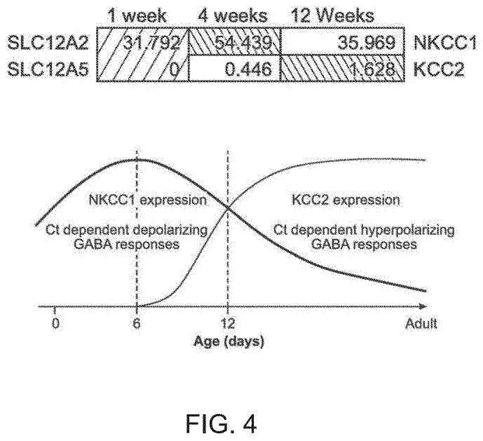

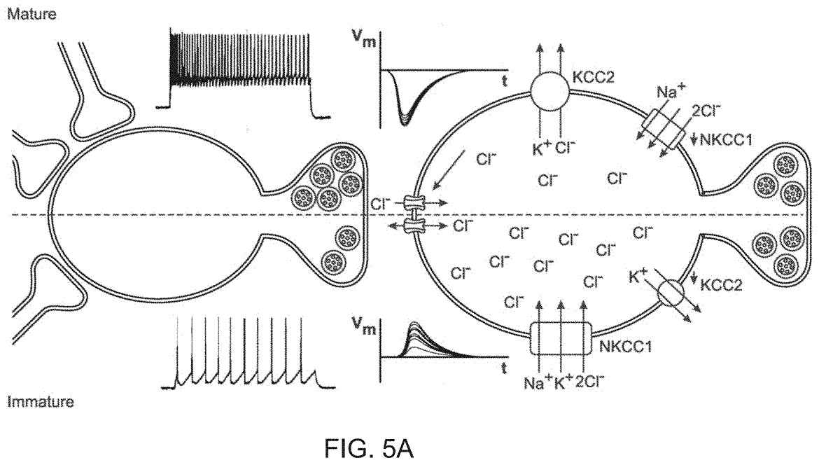

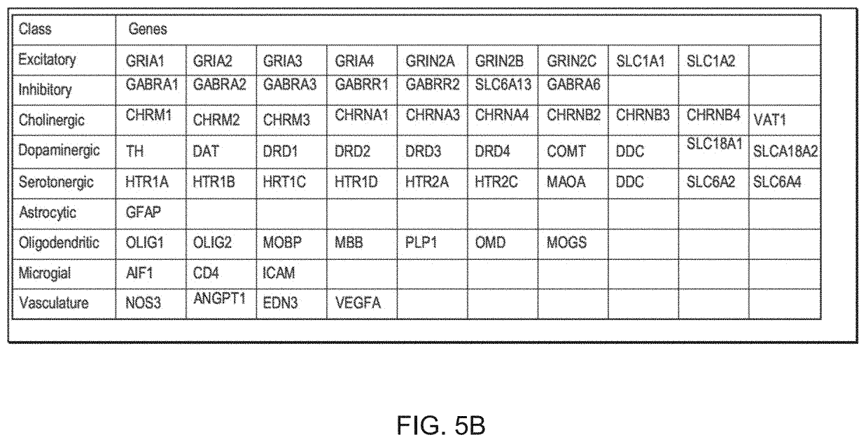

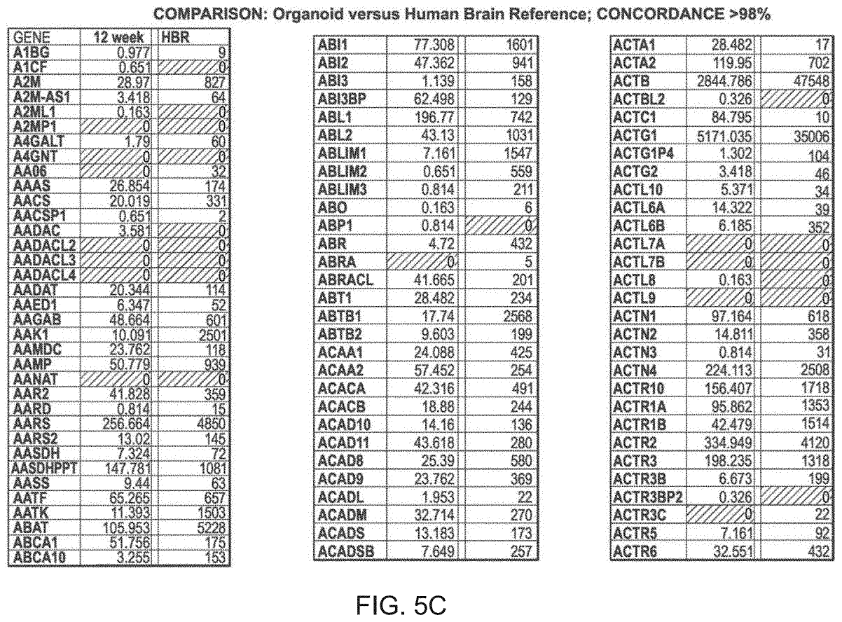

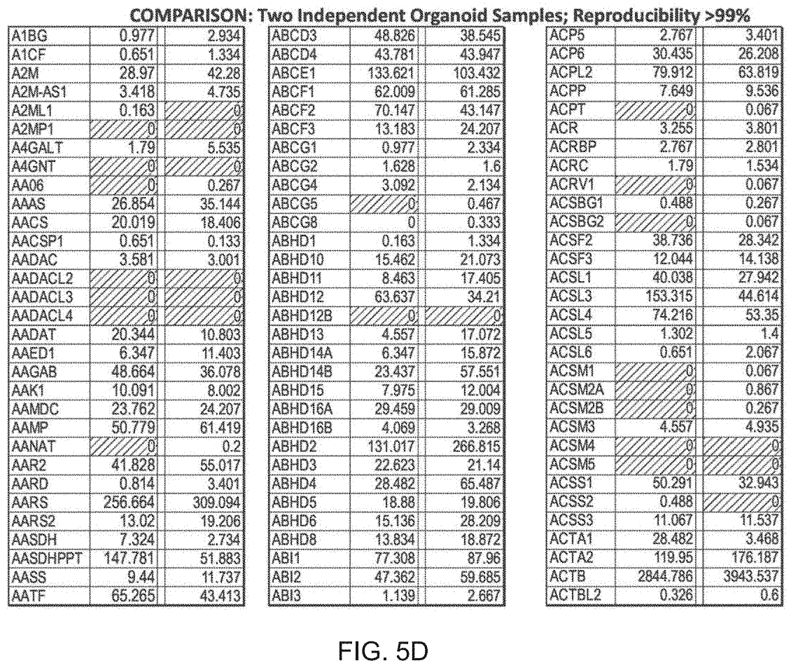

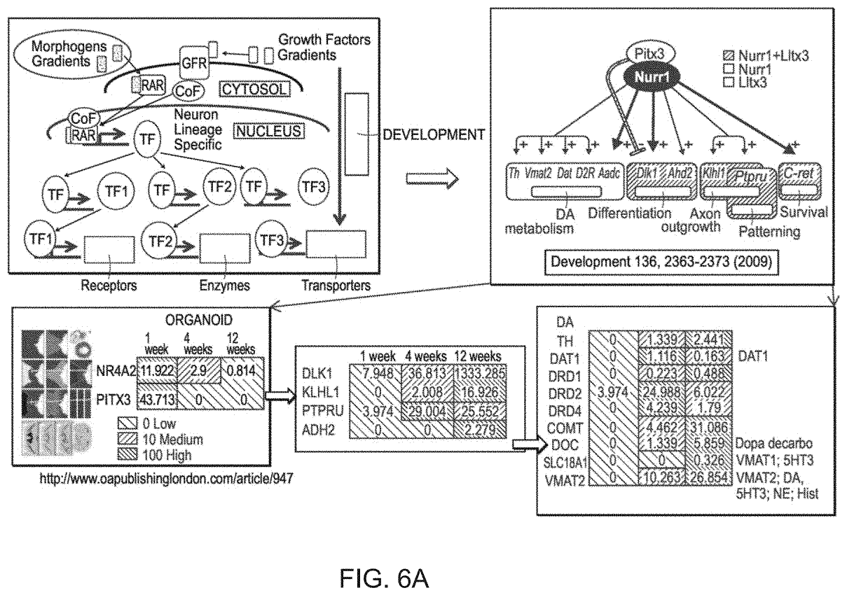

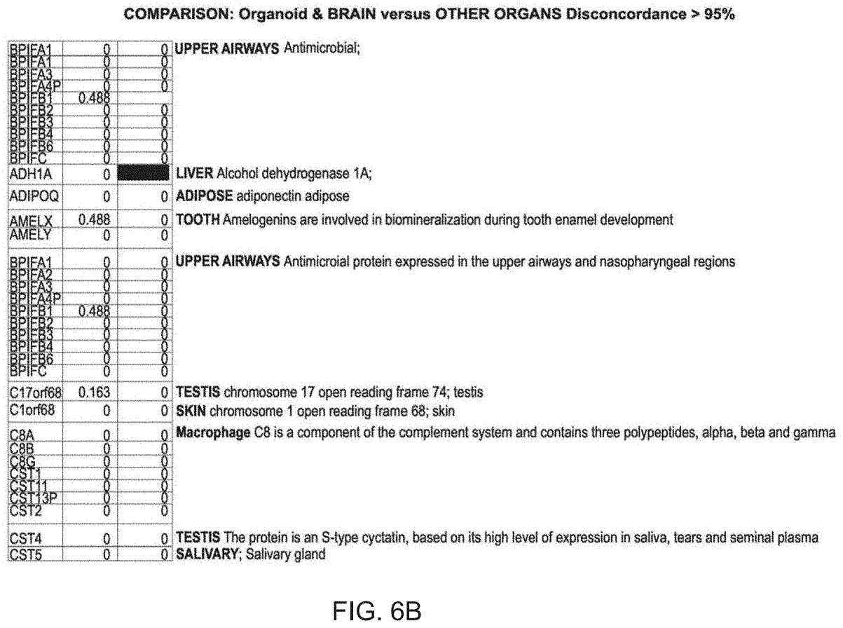

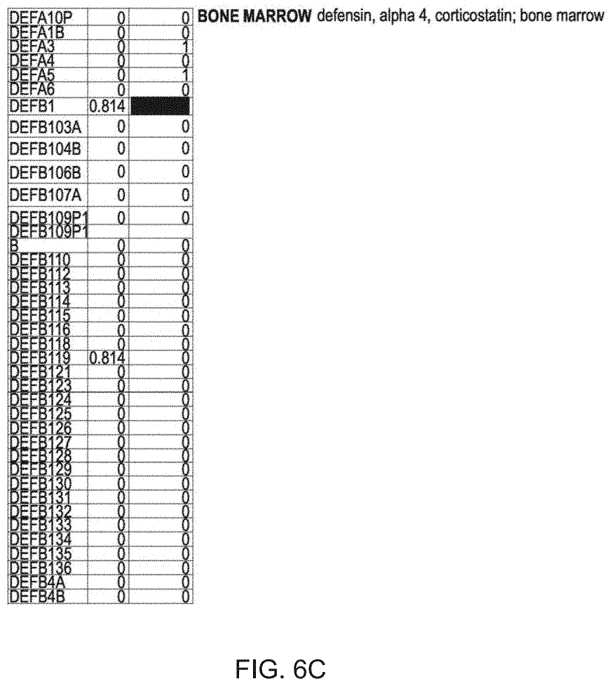

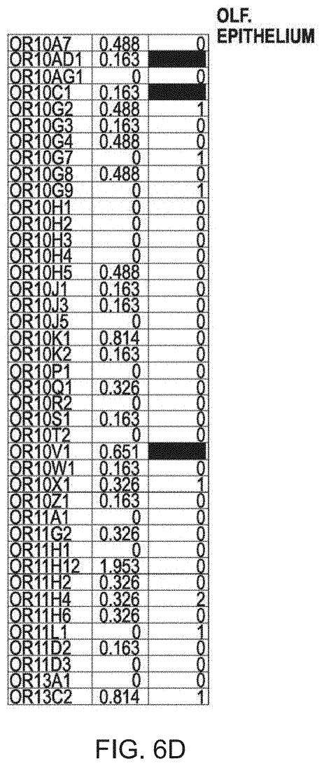

All human neural organoids were derived from iPSCs of fibroblast origin (from System Biosciences, Inc). The development of a variety of brain structures was characterized in the organoids. Retinal markers are shown in FIG. 15. Doublecortin (DCX), a microtubule associated protein expressed during cortical development, was observed in the human neural organoid (FIG. 1A and FIG. 1B, and FIG. 16). Midbrain development was characterized by the presence of tyrosine hydroxylase (FIG. 2). In addition, transcriptomics revealed expression of the midbrain markers DLKI, KLHL I, and PTPRU (FIG. 6A). GFAP staining was used to identify the presence of astrocytes in the organoids (FIG. 3). NeuN positive staining indicated the presence of mature neurons (FIG. 3). In addition, the presence of NKCCI and KCC2, neuron-specific membrane proteins, was observed in the organoid (FIG. 4). A schematic of the roles of NKCCI and KCC2 is provided in FIG. 5A. FIG. 5B indicates that a variety of markers expressed during human brain development are also expressed in the organoids described in Example 1.

Markers expressed within the organoids were consistent with the presence of excitatory, inhibitory, cholinergic, dopaminergic, serotonergic, astrocytic, oligodendritic, microglial, vasculature cell types. Further, the markers were consistent with those identified by the Human Brain Reference (HBR) from Clontech (FIG. 5C) and were reproducible in independent experiments (FIG. 5D). Non-brain tissue markers were not observed in the neural organoid (FIG. 6B).

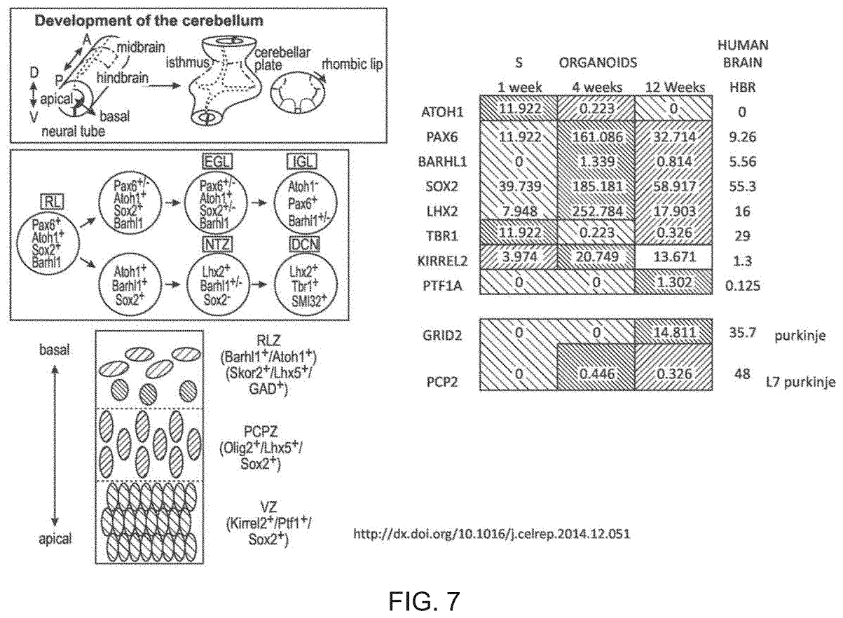

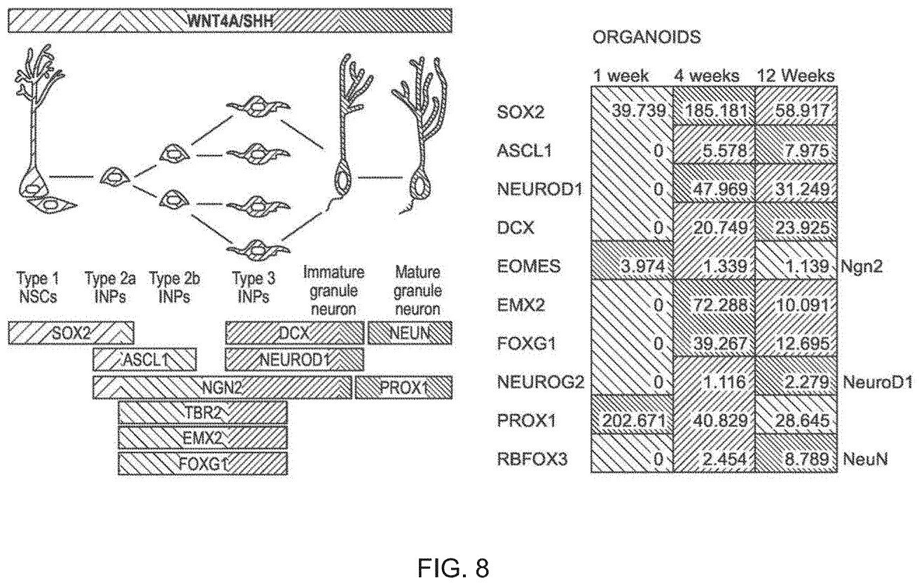

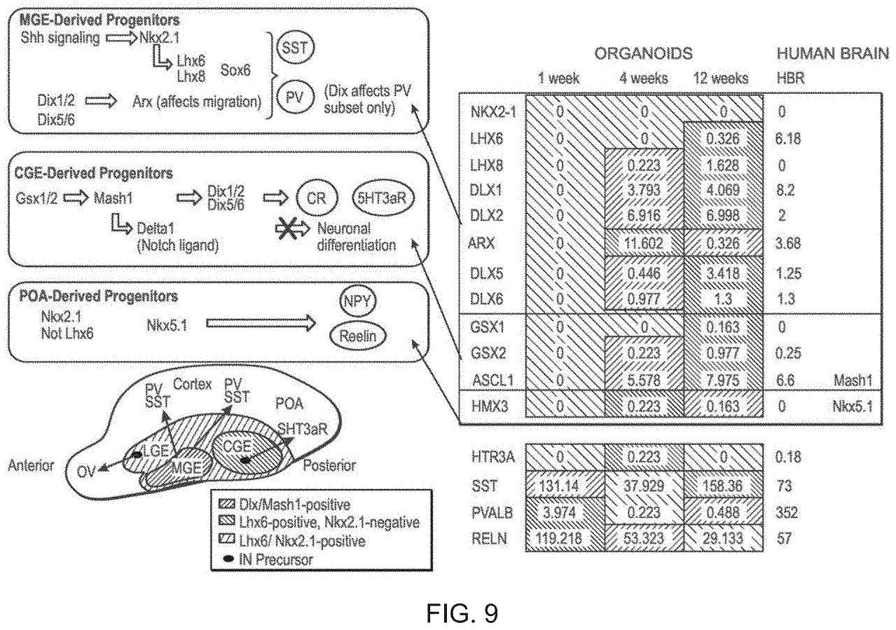

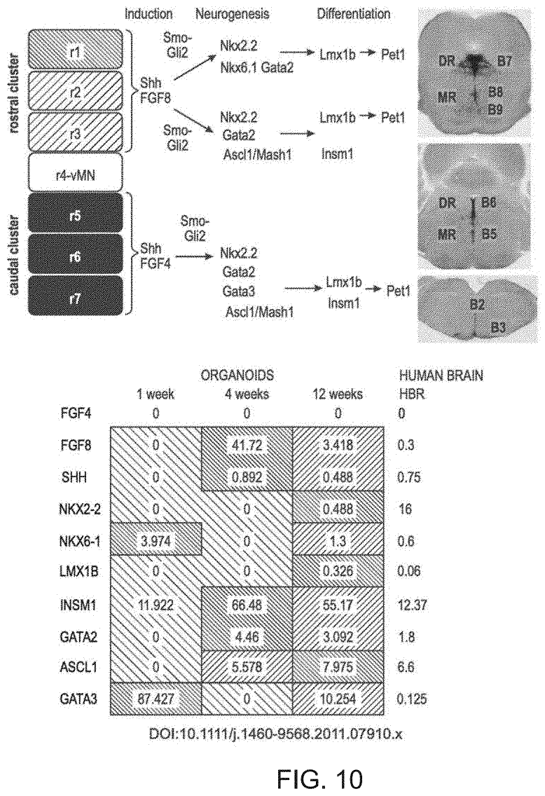

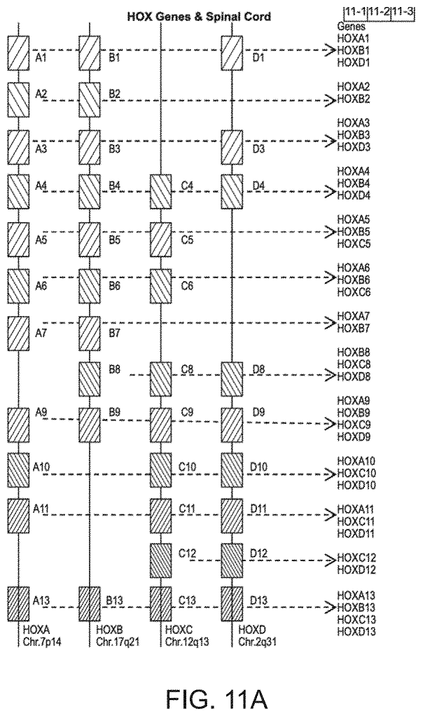

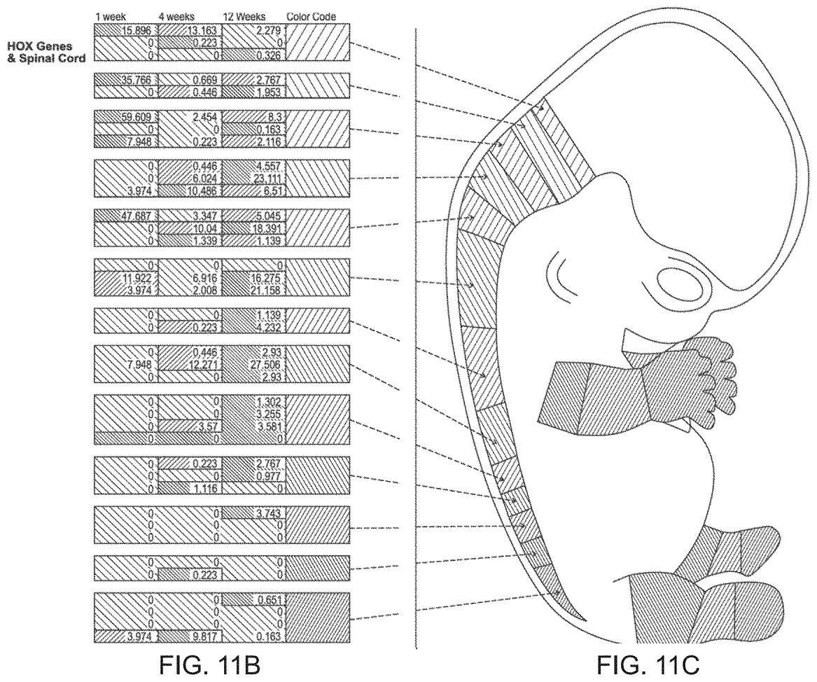

Tyrosine hydroxylase, an enzyme used in the synthesis of dopamine, was observed in the organoids using immunocytochemistry (FIG. 5B) and transcriptomics (FIG. 6A). The expression of other dopaminergic markers, including vesicular monoamine transporter 2 (VMAT2), dopamine active transporter (DAT) and dopamine receptor D2 (D2R) were observed using transcriptomic analysis. FIG. 7 delineates the expression of markers characteristic of cerebellar development. FIG. 8 provides a list of markers identified using transcriptomics that are characteristic of neurons present in the hippocampus dentate gyrus. Markers characteristic of the spinal cord were observed after 12 weeks of in vitro culture. FIG. 9 provides a list of markers identified using transcriptomics that are characteristic of GABAergic interneuron development. FIG. 10 provides a list of markers identified using transcriptomics that are characteristic of the brain stem, in particular, markers associated with the serotonergic raphe nucleus of the pons. FIG. 11 lists the expression of various Hox genes that are expressed during the development of the cervical, thoracic and lumbar regions of the spinal cord.

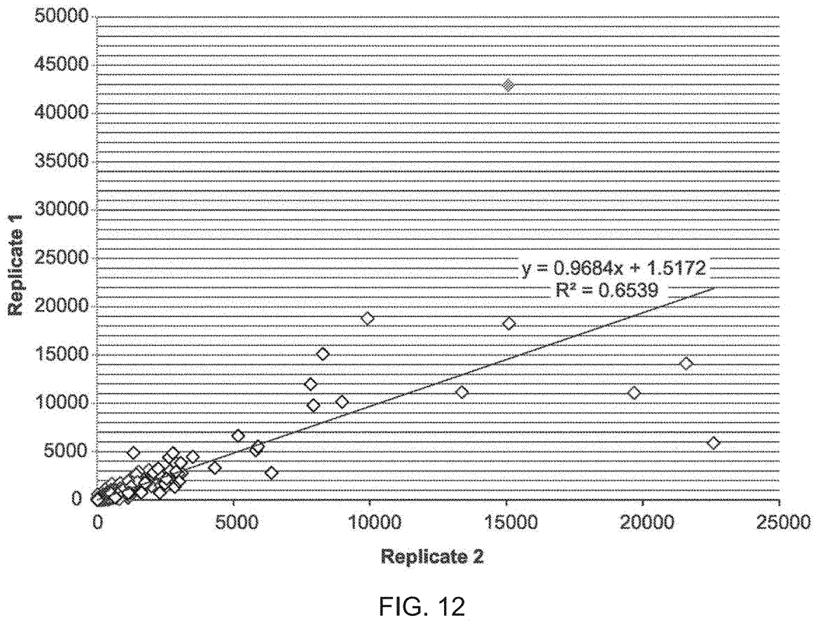

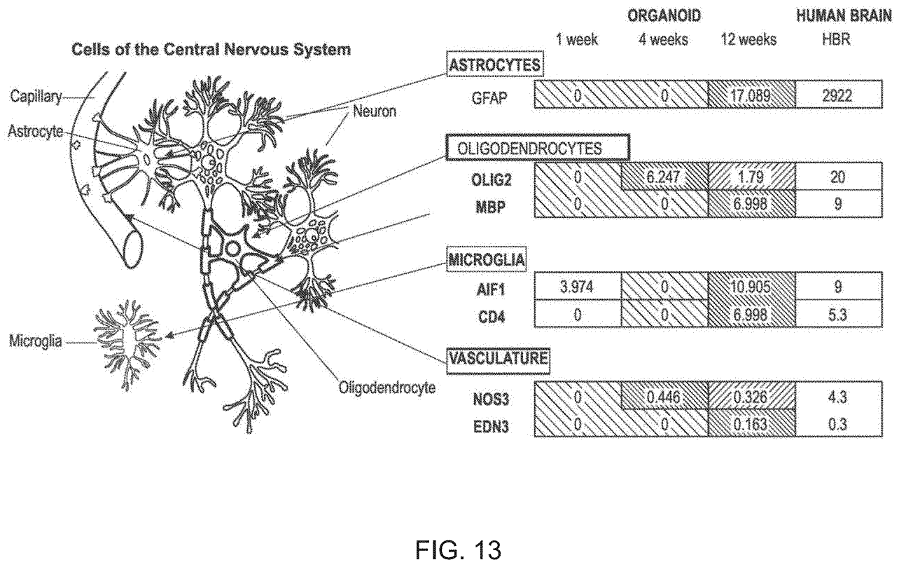

FIG. 12 shows that results are reproducible between experiments. The expression of markers detected using transcriptomics is summarized in FIG. 13.

In sum, the results reported herein support the conclusion that the invention provides an in vitro cultured organoid that resembles an approximately 5-week old human fetal brain, based on size and specific morphological features with great likeness to the optical stock, the cerebral hemisphere, and cephalic flexure in a 2-3mm organoid that can be grown in culture. High resolution morphology analysis was carried out using immunohistological methods on sections and confocal imaging of the organoid to establish the presence of neurons, axons, dendrites, laminar development of cortex, and the presence of midbrain marker.

This organoid includes an interactive milieu of brain circuits as represented by the laminar organization of the cortical structures in FIG. 13 and thus supports formation of native neural niches in which exchange of miRNA and proteins by exosomes can occur among different cell types.

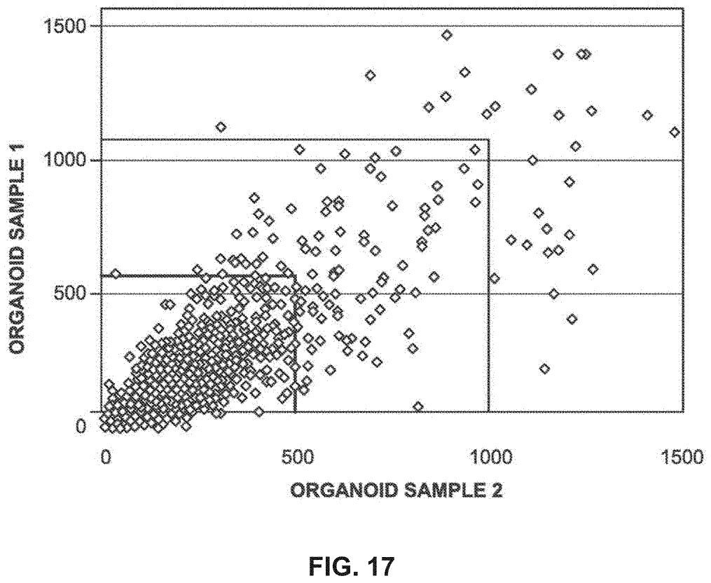

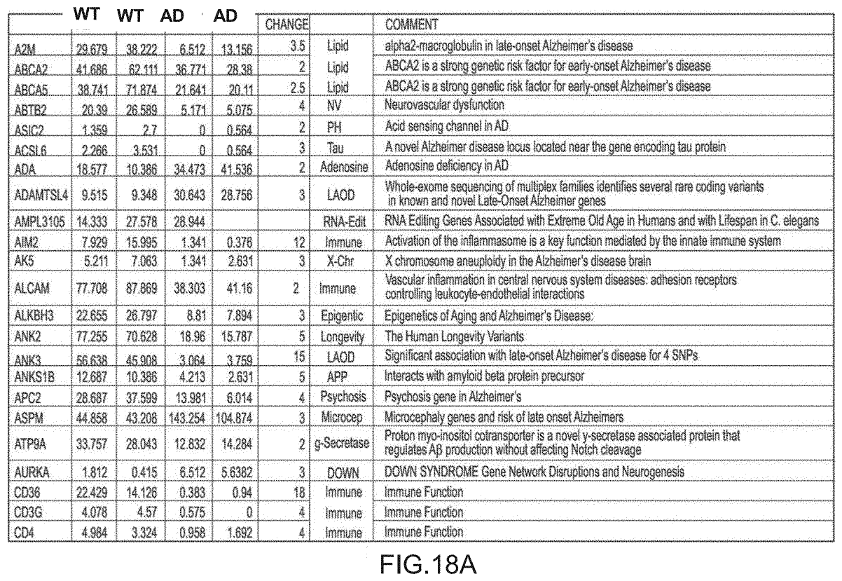

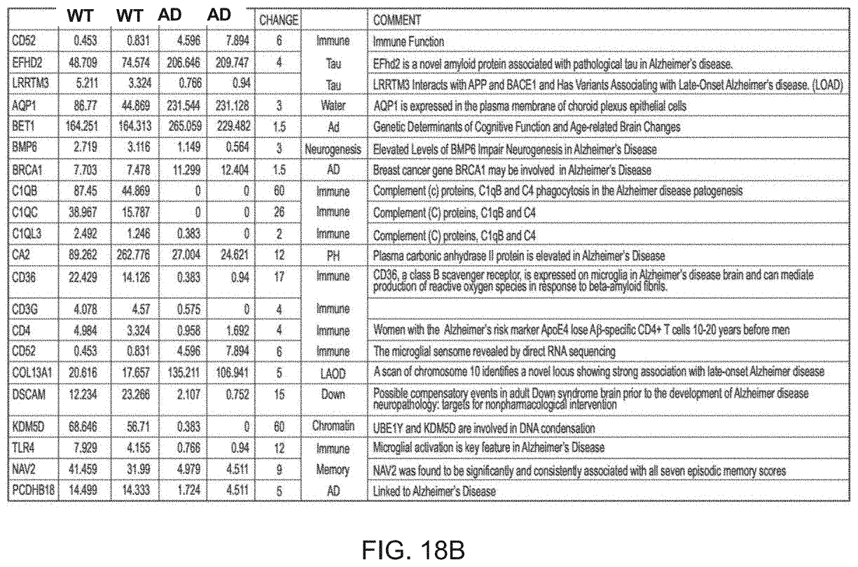

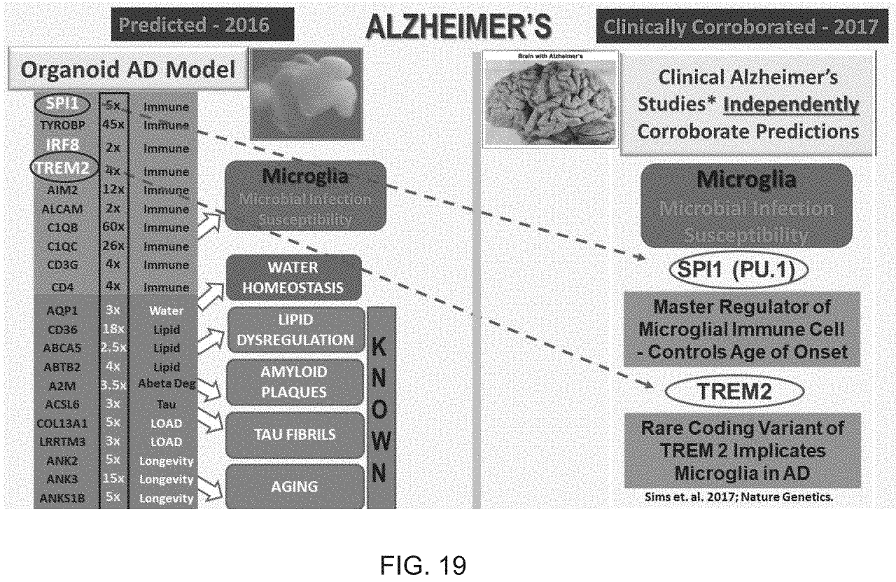

Neural organoids were evaluated at weeks 1, 4 and 12 by transcriptomics. The organoid was reproducible and replicable (FIGS. 5C, 5D, FIG. 12, and FIG. 17). Brain organoids generated in two independent experiments and subjected to transcriptomic analysis showed >99% replicability of the expression pattern and comparable expression levels of most genes with <2-fold variance among some of the replicates.

Gene expression patterns were analyzed using whole genome transcriptomics as a function of time in culture. Results reported herein indicate that within the neural organoid known developmental order of gene expression in vivo occurs, but on a somewhat slower timeline. For example, the in vitro temporal expression of the transcription factors NURRI and PITX3, genes uniquely expressed during midbrain development, replicated known in vivo gene expression patterns (FIG. 6A). Similarly, the transition from GABA mediating excitation to inhibition, occurred following the switch of the expression of the Na(⁺)-K(⁺)-2C1(⁻⁻)) cotransporter NKCCI (SLC12A2), which increases intracellular chloride ions, to the K(⁺)-Cl(⁻) cotransporter KCC2 (SLC12A5) (Owens and Kriegstein, Is there more to GABA than synaptic inhibition?, Nat Rev Neurosci. 3(9):715-27 2002), which decreases intracellular chloride ion concentrations (Blaesse et al., Cation-chloride cotransporters and neuronal function. Neuron. 61(6) 820-838, 2009). Data on the development of the brain organoids in culture showed that expression profiles of NKCCI and KCC2 changed in a manner consistent with an embryonic brain transitioning from GABA being excitatory to inhibitory (FIGS. 4 & 5), a change that can be monitored by developmental transcriptomics.

### Example 3

### Tuberous Sclerosis Complex (TSC) Model

Tuberous sclerosis complex (TSC) is a genetic disorder that causes non-malignant tumors to form in multiple organs, including the brain. TSC negatively affects quality of life, with patients experiencing seizures, developmental delay, intellectual disability, gastrointestinal distress and Alzheimer's disease. Two genes are associated with TSC: (1) the TSC1 gene, located on chromosome 9 and also referred to as the hamartin gene and (2) the TSC2 gene located on chromosome 16 and referred to as the tuberin gene.

Using methods as set forth in Example 1, a human neural organoid from iPSCs was derived from a patient with a gene variant of the TSC2 gene (ARG 1743GLN) from iPSCs (Cat# GM25318 Coriell Institute Repository, N.J.). This organoid served as a genetic model of a TSC2 mutant.

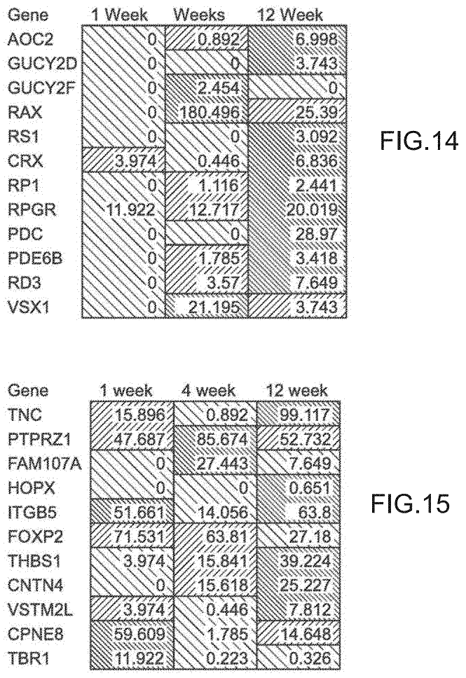

Both normal and TSC2 mutant models were subject to genome-wide transcriptomic analysis using the Ampliseg™ analysis (ThermoFisher) to assess changes in gene expression and how well changes correlated with the known TSC clinical pathology (FIG. 14).

Whole genome transcriptomic data showed that of all the genes expressed (13,000), less than a dozen showed greater than two-fold variance in the replicates for both Normal N)) and TSC2. This data supported the robustness and replicability of the human neural organoids at week 1 in culture.

Clinically TSC patients present with tumors in multiple organs including the brain, lungs, heart, kidneys and skin (Harmatomas). In comparison of WT and TSC2, the genes expressed at two-fold to 300-fold differences, included those correlated with 1) tumor formation and 2) Alzheimer's disease mapped using whole genome and exome sequencing strategies

### Example 4

### Human Neural Organoid Model Gene Expression to Predict Drug Abuse

Drug abuse susceptibility has a strong genetic link with DNA mutations comprising a common molecular characteristic of drug abuse susceptibility. The genes identified as playing a role in drug abuse susceptibility include novel markers provided in Table 1.

Expression changes and mutations in the noted genes disclosed herein from the neural organoid at about week 1, about week 4 and about week 12 are used in one embodiment to predict drug abuse susceptibility risk. In a further aspect, mutations in the genes disclosed can be determined at hours, days or weeks after reprogramming.

In a second embodiment, mutations in Table 1, in the human neural organoid at about week 1, about week 4, and about week 12 are used to predict drug abuse susceptibility using above described methods for calculating risk. One skilled in the art would recognize that additional biomarker combinations expressed in the human neural organoid can also be used to predict drug abuse susceptibility and onset.

The model used herein is validated and novel in that it uses, as starting material, an individual's iPSCs originating from skin or blood cells as the starting material to develop a neural organoid that allows for identification of drug abuse susceptibility markers early in development including at birth.

TABLE 4
Therapeutic Drug Abuse Susceptibility Biomarker Genes

| Gene | Clinical Comorbidity Susceptibility/Resistance |
|---|---|
| ABCB8 | Tick Infestation and Intestinal Tuberculosis. |
| ABCC2 | Dubin-Johnson Syndrome and Bilirubin Metabolic Disorder. |
| ADCY1 | Deafness, Autosomal Recessive 44 and Autosomal Recessive Non-Syndromic |
| | Sensorineural Deafness Type Dfnb. |
| ADCY7 | RET signaling and Oocyte meiosis. |
| ADHFE1 | D-2-Hydroxyglutaric Aciduria 1 TCA cycle |
| ADRBK2 | Cocaine Abuse. |
| ADPGK | Glycolysis, possibly during ischemic conditions |
| AKR1B1 | Diabetic Neuropathy and Diabetic Cataract. |
| ALDOC | Glucose metabolism and Innate Immune System |
| AMPH | Stiff-Person Syndrome and Limbic Encephalitis. |
| APOD | Breast Cyst and Niemann-Pick Disease. |
| ARL6IP4 | Infection by Herpes simplex virus (HSVI), may act as a splicing inhibitor of HSVI |
| | pre-mRNA. |
| ASAH1 | This enzyme is overexpressed in multiple human cancers and may play a role in |
| | cancer progression. |
| ASGR2 | The asialoglycoprotein receptor may facilitate hepatic infection by multiple |
| | viruses including hepatitis B |
| ATP13A4 | Smoking related |
| ATP1A2 | Smoking related |
| ATP5B | Pyelonephritis |
| NCR3LG1 | Immunoregulatory interactions between a Lymphoid and a non-Lymphoid cell |
| | and Innate Immune System. |
| BCL6 | Dopamine Receptor-Interacting Protein 1 |
| C14orf28 | DRIP-1; |
| ARL6IP4 | Herpes simplex virus (HSVI), |
| ASAH1 | regulator of steroidogenesis |
| ASGR2 | Hepatitis B. |
| ATF5 | West Nile Fever and Triple-Receptor Negative Breast Cancer. |
| ATL3 | Neuropathy, Hereditary Sensory, Type If and Hereditary Sensory And Autonomic |
| | Neuropathy Type 1 |
| NCR3LG1 | Selectively expressed on tumor cells. |
| BCL6 | Primary Mediastinal Large B-Cell Lymphoma and Intravascular Large B-Cell |
| | Lymphoma. |
| BICD1 | Lissencephaly 1. |
| ARL14EP | May connect MHC class II-containing cytoplasmic vesicles to the actin network |
| CASP3 | Helicobacter Pylori Infection and Fixed Drug Eruption. |
| CEBPD | Macrophage and regulation of genes involved in immune and inflammatory |
| | responses |
| CHN2 | Associated with schizophrenia in men |
| CLCN3 | Cystic Fibrosis. |
| CLDN7 | Ductal Carcinoma In Situ and Chromophobe Renal Cell Carcinoma. |
| CLOCK | Circadian |
| COL1A2 | Osteogenesis Imperfecta, Type Iii and Osteogenesis Imperfecta, Type Iv. |
| CRABP1 | Teratocarcinoma and Embryonal Carcinoma. |
| CRABP2 | Embryonal Carcinoma and Basal Cell Carcinoma. |
| CREM | Oligoarticular Juvenile Idiopathic Arthritis and Female Stress Incontinence. |
| CRHBP | Autosomal Dominant Nocturnal Frontal Lobe Epilepsy |
| CRY1 | Circadian |
| CRYAA | Cataract 9, Multiple Types and Cataract Microcornea Syndrome. |
| CRYAB | Myopathy, Myofibrillar, 2 and Cataract 16, Multiple Types. |
| CRYM | Deafness, Autosomal Dominant 40 and Retinitis Pigmentosa 22. |
| CTGF | https://www.ncbi.nlm.nih.gov/pmc/articles/PMC3058191/Limited Scleroderma |
| | and Systemic Scleroderma. |
| CTNNB1 | Pilomatrixoma and Neurodevelopmental Disorder With Spastic Diplegia And |
| | Visual Defects. |
| CTNND2 | Benign Adult Familial Myoclonic Epilepsy and Cri-Du-Chat Syndrome. |
| CCKBR | Panic Disorder and Anxiety. |
| DCC | Mirror Movements 1 and Gaze Palsy, Familial Horizontal, With Progressive |
| | Scoliosis 2, with Impaired Intellectual Development |
| CUX2 | TF; Regulates dendrite development and branching, dendritic spine formation, |
| | and synaptogenesis in cortical layers II-III |
| DDIT4 | Required for mTORC1-mediated defense against viral protein synthesis and |
| | virus replication (By similarity). Inhibits neuronal differentiation and neurite |
| | outgrowth mediated by NGF via its effect on mTORC1 activity. Required for |
| | normal neuron migration during embryonic brain development. Plays a role in |
| | neuronal cell death. |
| DLX1 | TF; differentiation of interneurons, such as amacrine and bipolar cells in the |
| | developing retina development of the ventral forebrain |
| DNAJB5 | Master negative regulator of cardiac hypertrophy |
| DPP7 | Innate Immune System. |
| DRD2 | Cocaine Dependence. |
| DUSP1 | Amyotrophic Lateral Sclerosis 7 and Amyotrophic Lateral Sclerosis 9. |
| DUSP6 | PAIN Dual Specificity Phosphatase 6 alleviating chronic postoperative pain |
| EGR1 | Circadian C2H2-type zinc-finger proteins rhythmic expression of core-clock gene |
| | ARNTL/BMAL1 in the suprachiasmatic nucleus |
| EMX2OS | Non-coding RNA class diseases associated with EMX2OS include Hypertension, |
| | Essential |
| ENO1 | Hashimoto Encephalopathy and Cancer-Associated Retinopathy. |
| ENO2 | Granular Cell Tumor and Small Cell Carcinoma. |
| ENO3 | Glucose metabolism and HIF-1 signaling pathway. |
| ENPP2 | Motility-related processes such as angiogenesis and neurite outgrowth. Acts as |
| | an angiogenic factor |
| EP300 | Histone acetyltransferase and regulates transcription |
| | HIV-1 infection, it is recruited by the viral protein Tat |
| | Participates in CLOCK or NPAS2-regulated rhythmic gene transcription; exhibits |
| | a circadian association with CLOCK or NPAS2, correlating with increase in |
| | PER1/2 mRNA and histone H3 acetylation on the PER1/2 promoter |
| ERRFI1 | Progesterone Resistance and Atrial Heart Septal Defect. |
| FAAH | Polysubstance Abuse and Cannabis Dependence. |
| FABP7 | Establishment of the radial glial fiber in the developing brain; necessary for the |
| | migration of immature neurons to establish cortical layers |
| FAM107A | Brain Cancer. |
| FBXL3 | CIRCADIAN; ubiquitin protein ligase complex maintenance of both the speed |
| | and the robustness of the circadian clock oscillation |
| FBXO45 | Control synaptic activity by controlling UNC13A via ubiquitin dependent pathway |
| FIGNL1 | DNA double-strand break repair via homologous recombination |
| FN1 | Glomerulopathy With Fibronectin Deposits 2 and Spondylometaphyseal |
| | Dysplasia, Corner Fracture Type. |
| FUBP1 | MYC; activator and repressor of transcription. |
| GAA | Glycogen Storage Disease Ii and Glycogen Storage Disease. |
| GABBR1 | Brain disorders such as schizophrenia and epilepsy. |
| GABRB1 | Pathogenetics of schizophrenia (GABA) A receptor |
| GABRB3 | Angelman syndrome, Prader-Willi syndrome, nonsyndromic orofacial clefts, |
| | epilepsy and autism |
| GAD1 | Role in the stiff man syndrome. |
| GCLC | Gamma-Glutamylcysteine Synthetase Deficiency, Hemolytic Anemia Due To |
| | and Myocardial Infarction |
| GLRA1 | (AMPA) receptors |
| GLRB | Hyperekplexia 2 and Hyperekplexia |
| GNAO1 | Drug Cue induced craving |
| GNAQ | Drug Cue induced craving |
| GRB2 | Adapter protein that provides a critical link between cell surface growth factor |
| | receptors and the Ras signaling pathway. |
| GRIA1 | (AMPA) receptors |
| GRIK5 | GRIK5 include Schizophrenia Glutamate Ionotropic Receptor Kainate |
| GRIN2A | (NMDA) receptor subunit |
| GRM3 | Schizophreniform Disorder and Schizophrenia; Glutamate Metabotropic |
| | Receptor 3 |
| HDAC4 | Initiation of transcription and translation elongation at the HIV-1 LTR. |
| HDAC5 | Initiation of transcription and translation elongation at the HIV-1 LTR |
| HIF1A | Hypoxia and Retinal Ischemia. |
| HIF3A | Tobacco Smoking |
| HIP1 | Clathrin-mediated endocytosis and trafficking |
| HMOX1 | Heme Oxygenase 1 Deficiency and Pulmonary Disease, Chronic Obstructive. |
| HOMER2 | Regulate group 1 metabotrophic glutamate receptor |
| HTR2A | Major Depressive Disorder and Obsessive-Compulsive Disorder |
| HTRA1 | Age-related macular degeneration type 7 |
| IGFBP7 | Tobacco Smoking |
| ITM2A | Osteo- and chondrogenic differentiation |
| ITPKB | Inositol phosphate metabolism |
| KALRN | Coronary Heart Disease 5 and Keratomalacia. |
| LMX1A | LIM Homeobox Transcription |
| | Factor 1 Alpha development of dopamine producing neurons during |
| | embryogenesis. |
| LRCH4 | |
| LYZ | Amyloidosis, Familial Visceral and Al Amyloidosis. |
| MAL | Vesicular trafficking cycling between the Golgi complex and the apical plasma |
| | membrane |
| MAOB | Oxidation of monoamines such as dopamine, serotonin and adrenalin |
| MPDZ | Control of AMPAR potentiation and synaptic plasticity in excitatory |
| | synapses; interact with the HTR2C receptor and may cause it to clump at the cell |
| | surface |
| MPZ | Schwann cells of the peripheral nervous system |
| MT1X | Metallothioneins |
| MT2A | Scrapie and Xeroderma Pigmentosum, Complementation Group B. |
| NCOA7 | Transcriptional activities |
| | Aryl Hydrocarbon Receptor and AHR Pathway. |
| NDUFS1 | Respiratory electron transport, ATP synthesis by chemiosmotic coupling, and |
| | heat production by uncoupling proteins. |
| NDUFV2 | Mitochondrial Complex I Deficiency, Nuclear Type 7 and Leigh Syndrome With |
| | Leukodystrophy. |
| NEFH | Charcot-Marie-Tooth Disease, Axonal, Type 2Cc and Amyotrophic Lateral |
| | Sclerosis 1 |
| NEFL | Charcot-Marie-Tooth Disease, Demyelinating, Type 1F and Charcot-Marie-Tooth |
| | Disease, Dominant Intermediate G. |
| NET1 | Oppositional Defiant Disorder and Breast Cancer. |
| NFKBIA | Inflammatory responses |
| NPY1R | Body Mass Index Quantitative Trait Locus 11 |
| NR1D1 | represses the circadian clock transcription factor aryl hydrocarbon receptor |
| | nuclear translocator-like protein 1 (ARNTL) |
| NR3C1 | Glucocorticoid receptor, which can function both as a transcription factor; |
| | pituitary gland |
| NR3C2 | Mineralocorticoid receptors (MRs) are nuclear hormone receptors |
| NR4A1 | Opioid addiction MICROGLIA |
| NR4A2 | Opioid addiction |
| NRGN | NRGN is a direct target for thyroid hormone in human brain; Hypothyroidism; |
| | Poor ability to tolerate cold, a feeling of tiredness, constipation, depression, and |
| | weight gain |
| NSF | Tetanus and Neuronal Intranuclear Inclusion Disease |
| NTRK2 | Glia neutrophin-dependent calcium signaling in glial cells and mediate |
| | communication between neurons and glia. |
| OPRK1 | Morphine Dependence and Alcohol Dependence. |
| OSBPL1A | Cocaine and Heroin |
| PCP4 | Neuronal differentiation through activation of calmodulin-dependent kinase |
| | signaling pathways |
| PDIA3 | Maxillary Sinus Squamous Cell Carcinoma and Anomalous Left Coronary Artery |
| | From The Pulmonary Artery |
| PDK4 | Platelet Glycoprotein Iv Deficiency and Diabetes Mellitus, Noninsulin-Dependent. |
| PER1 | Circadian |
| PER2 | Circadian |
| PER3 | Circadian |
| BLOC1S6 | Hermansky-Pudlak Syndrome 9 |
| PLOD2 | Tobacco Smoking |
| PMP22 | Charcot-Marie-Tooth Disease And Deafness and Charcot-Marie-Tooth Disease, |
| | Demyelinating, Type 1A |
| PPP1R2 | Cataract 11, Multiple Types and Congenital Stationary Night Blindness. |
| PREX2 | Tobacco Smoking |
| PRKCE | Rift Valley Fever and Streptococcus Pneumonia |
| PTN | Tobacco Smoking |
| PURA | Mental Retardation, Autosomal Dominant 31 |
| RGS17 | Substance Dependence |
| RGS20 | Signaling by GPCR and Activation of cAMP-Dependent PKA. |
| RHOU | Signaling by Rho GTPases and Innate Immune System. |
| RNF125 | Tenorio Syndrome. |
| RPS6KA5 | Activated TLR4 signaling and Bladder cancer. |
| RPSA | Asplenia, Isolated Congenital and Venezuelan Equine Encephalitis |
| S1PR1 | Tobacco Smoking |
| SESN1 | Maxillary Cancer |
| SF3B1 | Myelodysplastic Syndrome and Autosomal Recessive Pyridoxine-Refractory |
| | Sideroblastic Anemia 2. |
| SGK3 | Breast Cancer |
| SLC1A2 | Tobacco Smoking |
| SLC1A3 | Tobacco Smoking |
| SLC3A2 | Tobacco Smoking |
| SLC4A4 | Tobacco Smoking |
| SLC4A7 | Renal Tubular Acidosis and Usher Syndrome, Type Iiia. |
| SLC7A11 | Tobacco Smoking |
| SMPD2 | Lipid Storage Disease and Coffin-Siris Syndrome 1 |
| SNAP25 | Myasthenic Syndrome, Congenital, 18 and Presynaptic Congenital Myasthenic |
| | Syndromes. |
| SOX2 | Heroin |
| SPTBN2 | Spinocerebellar Ataxia 5 and Spinocerebellar Ataxia, Autosomal Recessive 14. |
| STX1A | Cystic Fibrosis and Osteogenesis Imperfecta, Type Xv. |
| STXBP1 | Early Infantile Epileptic Encephalopathy |
| STXBP2 | Hemophagocytic Lymphohistiocytosis |
| SV2A | Alcohol-Related Birth Defect |
| SYN2 | Schizophrenia and Bipolar Disorder |
| SYT1 | Cocaine and Heroine |
| TAC1 | Heroin |
| TACSTD2 | Carcinoma-associated antigen. Tumor Associated Calcium Signal. Transducer |
| | 2 |
| TAGLN3 | Actin filament binding |
| TF | Atransferrinemia and Iron Deficiency Anemia. |
| TFAP2B | AP-2 family of transcription factors |
| TFRC | Immunodeficiency 46 and Combined Immunodeficiency, X-Linked. |
| TH | Conversion of tyrosine to dopamine |
| TIMP1 | Cocaine and heroin |
| TP53BP2 | Tobacco Smoking |
| TPH2 | Serotonin Major Depressive Disorder and Depression. |
| TRIB2 | Wnt/Hedgehog/Notch and DNA Damage; Uveitis and Narcolepsy. |
| TRIM59 | Drug cue induced |
| TRIP10 | Translocation of GLUT4 to the plasma membrane in response to insulin |
| | signaling; Wiskott-Aldrich Syndrome |
| TSPAN13 | Regulation of cell development, activation, growth and motility |
| TULP1 | Physiology of photoreceptors Leber Congenital Amaurosis 15 and Retinitis |
| | Pigmentosa 14. |
| VEGFA | Tobacco Smoking |
| WIF1 | Functions to inhibit WNT proteins; tumor suppressor gene, Esophageal Basaloid |
| | Squamous |
| ZBTB16 | Tobacco Smoking |
| ZMYM1 | Zinc Finger MYM-Type Containing 1 |
| LMO3 | Cysteine-rich LIM domain |
| | Oncogene member |

One of skill in the art will recognize that sequence data for the genes listed above can be obtained in publicly available gene databases such as GeneCards, GenBank, Malcard, Uniport and PathCard databases.

TABLE 5
Diagnostic Drug Abuse Susceptibility Biomarker Genes

| Gene | Clinical Comorbidity Susceptibility/Resistance |
|---|---|
| ABCB8 | Tick Infestation and Intestinal Tuberculosis. |
| ABCC2 | Dubin-Johnson Syndrome and Bilirubin Metabolic Disorder. |
| ADCY1 | Deafness, Autosomal Recessive 44 and Autosomal Recessive Non-Syndromic |
| | Sensorineural Deafness Type Dfnb. |
| ADCY7 | RET signaling and Oocyte meiosis. |
| ADHFE1 | D-2-Hydroxyglutaric Aciduria 1 TCA cycle |
| ADRBK2 | Cocaine Abuse. |
| ADPGK | Glycolysis, possibly during ischemic conditions |
| AKR1B1 | Diabetic Neuropathy and Diabetic Cataract. |
| ALDOC | Glucose metabolism and Innate Immune System |
| AMPH | Stiff-Person Syndrome and Limbic Encephalitis. |
| APOD | Breast Cyst and Niemann-Pick Disease. |
| ARL6IP4 | Infection by Herpes simplex virus (HSVI), may act as a splicing inhibitor of HSVI |
| | pre-mRNA. |
| ASAH1 | This enzyme is over-expressed in multiple human cancers and may play a role in |
| | cancer progression. |
| ASGR2 | The asialoglycoprotein receptor may facilitate hepatic infectionby multiple viruses |
| | including hepatitis B |
| ATP13A4 | Smoking related |
| ATP1A2 | Smoking related |
| ATP5B | Pyelonephritis |
| NCR3LG1 | Immunoregulatory interactions between a Lymphoid and a non-Lymphoid cell and |
| | Innate Immune System. |
| BCL6 | Dopamine Receptor-Interacting Protein 1 |
| C14orf28 | DRIP-1; |
| ARL6IP4 | Herpes simplex virus (HSVI), |
| ASAH1 | regulator of steroidogenesis |
| ASGR2 | Hepatitis B. |
| ATF5 | West Nile Fever and Triple-Receptor Negative Breast Cancer. |
| ATL3 | Neuropathy, Hereditary Sensory, Type If and Hereditary Sensory And Autonomic |
| | Neuropathy Type 1 |
| NCR3LG1 | Selectively expressed on tumor cells. |
| BCL6 | Primary Mediastinal Large B-Cell Lymphoma and Intravascular Large B-Cell |
| | Lymphoma. |
| BICD1 | Lissencephaly 1. |
| ARL14EP | May connect MHC class II-containing cytoplasmic vesicles to the actin network |
| CASP3 | Helicobacter Pylori Infection and Fixed Drug Eruption. |
| CEBPD | Macrophage and regulation of genes involved in immune and inflammatory |
| | responses |
| CHN2 | Associated with schizophrenia in men |
| CLCN3 | Cystic Fibrosis. |
| CLDN7 | Ductal Carcinoma In Situ and Chromophobe Renal Cell Carcinoma. |
| CLOCK | Circadian |
| COL1A2 | Osteogenesis Imperfecta, Type Iii and Osteogenesis Imperfecta, Type Iv. |
| CRABP1 | Teratocarcinoma and Embryonal Carcinoma. |
| CRABP2 | Embryonal Carcinoma and Basal Cell Carcinoma. |
| CREM | Oligoarticular Juvenile Idiopathic Arthritis and Female Stress Incontinence. |
| CRHBP | Autosomal Dominant Nocturnal Frontal Lobe Epilepsy |
| CRY1 | Circadian |
| CRYAA | Cataract 9, Multiple Types and Cataract Microcornea Syndrome. |
| CRYAB | Myopathy, Myofibrillar, 2 and Cataract 16, Multiple Types. |
| CRYM | Deafness, Autosomal Dominant 40 and Retinitis Pigmentosa 22. |
| CTGF | https://www.ncbi.nlm.nih.gov/pmc/articles/PMC3058191/Limited Scleroderma and |
| | Systemic Scleroderma. |
| CTNNB1 | Pilomatrixoma and Neurodevelopmental Disorder With Spastic Diplegia And Visual |
| | Defects. |
| CTNND2 | Benign Adult Familial Myoclonic Epilepsy and Cri-Du-Chat Syndrome. |
| CCKBR | Panic Disorder and Anxiety. |
| DCC | Mirror Movements 1 and Gaze Palsy, Familial Horizontal, With Progressive |
| | Scoliosis 2, |
| | With Impaired Intellectual Development |
| CUX2 | TF; Regulates dendrite development and branching, dendritic spine formation, and |
| | synaptogenesis in cortical layers II-III |
| DDIT4 | Required for mTORC1-mediated defense against viral protein synthesis and virus |
| | replication (By similarity). Inhibits neuronal differentiation and neurite outgrowth |
| | mediated by NGF via its effect on mTORC1 activity. Required for normal neuron |
| | migration during embryonic brain development. Plays a role in neuronal cell death. |
| DLX1 | TF; differentiation of interneurons, such as amacrine and bipolar cells in the |
| | developing retina development of the ventral forebrain |
| DNAJB5 | Master negative regulator of cardiac hypertrophy |
| DPP7 | Innate Immune System. |
| DRD2 | Cocaine Dependence. |
| DUSP1 | Amyotrophic Lateral Sclerosis 7 and Amyotrophic Lateral Sclerosis 9. |
| DUSP6 | PAIN Dual Specificity Phosphatase 6 alleviating chronic postoperative pain |
| EGR1 | Circadian C2H2-type zinc-finger proteins rhythmic expression of core-clock gene |
| | ARNTL/BMAL1 in the suprachiasmatic nucleus |
| EMX2OS | Non-coding RNA class diseases associated with EMX2OS include Hypertension, |
| | Essential |
| ENO1 | Hashimoto Encephalopathy and Cancer-Associated Retinopathy. |
| ENO2 | Granular Cell Tumor and Small Cell Carcinoma. |
| ENO3 | Glucose metabolism and HIF-1 signaling pathway. |
| ENPP2 | Motility-related processes such as angiogenesis and neurite outgrowth. Acts as an |
| | angiogenic factor |
| EP300 | Histone acetyltransferase and regulates transcription |
| | HIV-1 infection, it is recruited by the viral protein Tat |
| | Participates in CLOCK or NPAS2-regulated rhythmic gene transcription; exhibits a |
| | circadian association with CLOCK or NPAS2, correlating with increase in PER1/2 |
| | mRNA and histone H3 acetylation on the PER1/2 promoter |
| ERRFI1 | Progesterone Resistance and Atrial Heart Septal Defect. |
| FAAH | Polysubstance Abuse and Cannabis Dependence. |
| FABP7 | Establishment of the radial glial fiber in the developing brain; necessary for the |
| | migration of immature neurons to establish cortical layers |
| FAM107A | Brain Cancer. |
| FBXL3 | CIRCADIAN; ubiquitin protein ligase complex maintenance of both the speed and |
| | the robustness of the circadian clock oscillation |
| FBXO45 | Control synaptic activity by controlling UNC13A via ubiquitin dependent pathway |
| FIGNL1 | DNA double-strand break repair via homologous recombination |
| FN1 | Glomerulopathy With Fibronectin Deposits 2 and Spondylometaphyseal Dysplasia, |
| | Corner Fracture Type. |
| FUBP1 | MYC; activator and repressor of transcription. |
| GAA | Glycogen Storage Disease Ii and Glycogen Storage Disease. |
| GABBR1 | Brain disorders such as schizophrenia and epilepsy. |
| GABRB1 | Pathogenetics of schizophrenia (GABA) A receptor |
| GABRB3 | Angelman syndrome, Prader-Willi syndrome, nonsyndromic orofacial clefts, |
| | epilepsy and autism |
| GAD1 | Role in the stiff man syndrome. |
| GCLC | Gamma-Glutamylcysteine Synthetase Deficiency, Hemolytic Anemia Due To and |
| | Myocardial Infarction |
| GLRA1 | (AMPA) receptors |
| GLRB | Hyperekplexia 2 and Hyperekplexia. |
| GNAO1 | Drug Cue induced craving |
| GNAQ | Drug Cue induced craving |
| GRB2 | Adapter protein that provides a critical link between cell surface growth factor |
| | receptors and the Ras signaling pathway. |
| GRIA1 | (AMPA) receptors |
| GRIK5 | GRIK5 include Schizophrenia Glutamate Ionotropic Receptor Kainate |
| GRIN2A | (NMDA) receptor subunit |
| GRM3 | Schizophreniform Disorder and Schizophrenia; Glutamate Metabotropic Receptor 3 |
| HDAC4 | Initiation of transcription and translation elongation at the HIV-1 LTR. |
| HDAC5 | Initiation of transcription and translation elongation at the HIV-1 LTR |
| HIF1A | Hypoxia and Retinal Ischemia. |
| HIF3A | Tobacco Smoking |
| HIP1 | Clathrin-mediated endocytosis and trafficking |
| HMOX1 | Heme Oxygenase 1 Deficiency and Pulmonary Disease, Chronic Obstructive. |
| HOMER2 | Regulate group 1 metabotrophic glutamate receptor |
| HTR2A | Major Depressive Disorder and Obsessive-Compulsive Disorder |
| HTRA1 | Age-related macular degeneration type 7 |
| IGFBP7 | Tobacco Smoking |
| ITM2A | Osteo- and chondrogenic differentiation |
| ITPKB | Inositol phosphate metabolism |
| KALRN | Coronary Heart Disease 5 and Keratomalacia. |
| LMX1A | LIM Homeobox Transcription |
| | Factor 1 Alpha development of dopamine producing neurons during |
| | embryogenesis. |
| LRCH4 | |
| LYZ | Amyloidosis, Familial Visceral and Al Amyloidosis. |
| MAL | Vesicular trafficking cycling between the Golgi complex and the apical plasma |
| | membrane |
| MAOB | Oxidation of monoamines such as dopamine, serotonin and adrenalin |
| MPDZ | Control of AMPAR potentiation and synaptic plasticity in excitatory synapses; |
| | interact with the HTR2C receptor and may cause it to clump at the cell surface |
| MPZ | Schwann cells of the peripheral nervous system |
| MT1X | Metallothioneins |
| MT2A | Scrapie and Xeroderma Pigmentosum, Complementation Group B. |
| NCOA7 | Transcriptional activities; Aryl Hydrocarbon Receptor and AHR Pathway. |
| NDUFS1 | Respiratory electron transport, ATP synthesis by chemiosmotic coupling, and heat |
| | production by uncoupling proteins. |
| NDUFV2 | Mitochondrial Complex I Deficiency, Nuclear Type 7 and Leigh Syndrome With |
| | Leukodystrophy. |
| NEFH | Charcot-Marie-Tooth Disease, Axonal, Type 2Cc and Amyotrophic Lateral |
| | Sclerosis 1 |
| NEFL | Charcot-Marie-Tooth Disease, Demyelinating, Type 1F and Charcot-Marie-Tooth |
| | Disease, Dominant Intermediate G. |
| NET1 | Oppositional Defiant Disorder and Breast Cancer. |
| NFKBIA | Inflammatory responses |
| NPY1R | Body Mass Index Quantitative Trait Locus 11 |
| NR1D1 | represses the circadian clock transcription factor aryl hydrocarbon receptor nuclear |
| | translocator-like protein 1 (ARNTL) |
| NR3C1 | Glucocorticoid receptor, which can function both as a transcription factor; |
| | pituitary gland |
| NR3C2 | Mineralocorticoid receptors (MRs) are nuclear hormone receptors |
| NR4A1 | Opioid addiction via microglia |
| NR4A2 | Opioid addiction |
| NRGN | NRGN is a direct target for thyroid hormone in human brain |
| | Hypothyroidism |
| | Poor ability to tolerate cold, a feeling of tiredness, constipation, depression, and |
| | weight gain |
| NSF | Tetanus and Neuronal Intranuclear Inclusion Disease |
| NTRK2 | Glia neutrophin-dependent calcium signaling in glial cells and mediate |
| | communication between neurons and glia. |
| OPRK1 | Morphine Dependence and Alcohol Dependence. |
| OSBPL1A | Cocaine and Heroin |
| PCP4 | Neuronal differentiation through activation of calmodulin-dependent kinase |
| | signaling pathways |
| PDIA3 | Maxillary Sinus Squamous Cell Carcinoma and Anomalous Left Coronary Artery |
| | From The Pulmonary Artery |
| PDK4 | Platelet Glycoprotein Iv Deficiency and Diabetes Mellitus, Noninsulin-Dependent. |
| PER1 | Circadian |
| PER2 | Circadian |
| PER3 | Circadian |
| BLOC1S6 | Hermansky-Pudlak Syndrome 9 |
| PLOD2 | Tobacco Smoking |
| PMP22 | Charcot-Marie-Tooth Disease And Deafness and Charcot-Marie-Tooth Disease, |
| | Demyelinating, Type 1A |
| PPP1R2 | Cataract 11, Multiple Types and Congenital Stationary Night Blindness. |
| PREX2 | Tobacco Smoking |
| PRKCE | Rift Valley Fever and Streptococcus Pneumonia |
| PTN | Tobacco Smoking |
| PURA | Mental Retardation, Autosomal Dominant 31 |
| RGS17 | Substance Dependence. |
| RGS20 | Signaling by GPCR and Activation of cAMP-Dependent PKA. |
| RHOU | Signaling by Rho GTPases and Innate Immune System. |
| RNF125 | Tenorio Syndrome. |
| RPS6KA5 | Activated TLR4 signaling and Bladder cancer. |
| RPSA | Asplenia, Isolated Congenital and Venezuelan Equine Encephalitis |
| S1PR1 | Tobacco Smoking |
| SESN1 | Maxillary Cancer |
| SF3B1 | Myelodysplastic Syndrome and Autosomal Recessive Pyridoxine-Refractory |
| | Sideroblastic Anemia 2. |
| SGK3 | Breast Cancer |
| SLC1A2 | Tobacco Smoking |
| SLC1A3 | Tobacco Smoking |
| SLC3A2 | Tobacco Smoking |
| SLC4A4 | Tobacco Smoking |
| SLC4A7 | Renal Tubular Acidosis and Usher Syndrome, Type Iiia. |
| SLC7A11 | Tobacco Smoking |
| SMPD2 | Lipid Storage Disease and Coffin-Siris Syndrome 1 |
| SNAP25 | Myasthenic Syndrome, Congenital, 18 and Presynaptic Congenital Myasthenic |
| | Syndromes. |
| SOX2 | Heroin |
| SPTBN2 | Spinocerebellar Ataxia 5 and Spinocerebellar Ataxia, Autosomal Recessive 14. |
| STX1A | Cystic Fibrosis and Osteogenesis Imperfecta, Type Xv. |
| STXBP1 | Early Infantile Epileptic Encephalopathy |
| STXBP2 | Hemophagocytic Lymphohistiocytosis |
| SV2A | Alcohol-Related Birth Defect |
| SYN2 | Schizophrenia and Bipolar Disorder |
| SYT1 | Cocaine and Heroine |
| TAC1 | Heroin |
| TACSTD2 | Carcinoma-associated antigen. Tumor Associated Calcium Signal Transducer 2 |
| TAGLN3 | Actin filament binding |
| TF | Atransferrinemia and Iron Deficiency Anemia. |
| TFAP2B | AP-2 family of transcription factors |
| TFRC | Immunodeficiency 46 and Combined Immunodeficiency, X-Linked. |
| TH | Conversion of tyrosine to dopamine |
| TIMP1 | Cocaine and heroin |
| TP53BP2 | Tobacco Smoking |
| TPH2 | Serotonin Major Depressive Disorder and Depression. |
| TRIB2 | Wnt/Hedgehog/Notch and DNA Damage; Uveitis and Narcolepsy. |
| TRIM59 | Drug cue induced |
| TRIP10 | Translocation of GLUT4 to the plasma membrane in response to insulin signaling; |
| | Wiskott-Aldrich Syndrome |
| TSPAN13 | Regulation of cell development, activation, growth and motility |
| TULP1 | Physiology of photoreceptors Leber Congenital Amaurosis 15 and Retinitis |
| | Pigmentosa 14. |
| VEGFA | Tobacco Smoking |
| WIF1 | Functions to inhibit WNT proteins; tumor suppressor gene, Esophageal Basaloid |
| | Squamous |
| ZBTB16 | Tobacco Smoking |
| ZMYM1 | Zinc Finger MYM-Type Containing 1 |
| LMO3 | Cysteine-rich LIM domain. Oncogene member |

One of skill in the art will recognize that sequence data for the genes listed above can be obtained in publicly available gene databases such as GeneCards, GenBank, Malcard, Uniport and PathCard databases.

### Example 5

### Predicting Risk of Disease Onset from Neural Organoid Gene Expression

Gene expression in the neural organoid can be used to predict disease onset. Briefly, gene expression is correlated with Gene Card and Pubmed database genes and expression compared for dysregulated expression in diseased vs non-disease neural organoid gene expression.

### Example 6

### Prediction of Co-Morbidities Associated with Drug Abuse and Drug Abuse Susceptibility

The human neural organoid model data findings can be used in the prediction of comorbidity onset or risk associated with substance abuse including at birth (Reference: European Bioinformatic Institute (EBI) and ALLEN INSTITUTE databases) and in detecting comorbidities, genes associated with one or more of these diseases are detected from the patient's grown neural organoid. Such genes include, comorbidities and related accession numbers include, those listed in Table 6

TABLE 6
Co-Morbidities Associated with Drug Abuse

| Gene | Clinical Susceptibility/Resistance |
|---|---|
| ABCB8 | Tick Infestation and Intestinal Tuberculosis |
| ABCC2 | Dubin-Johnson Syndrome |
| | Bilirubin Metabolic Disorder |
| ADCY1 | Deafness, Autosomal Recessive 44 and Autosomal Recessive Non-Syndromic |
| | Sensorineural Deafness Type Dfnb. |
| ADCY7 | RET signaling and Oocyte meiosis. |
| ADHFE1 | D-2-Hydroxyglutaric Aciduria 1 TCA cycle |
| ADPGK | Glycolysis, possibly during ischemic conditions |
| AKR1B1 | Diabetic Neuropathy and Diabetic Cataract |
| ALDOC | Glucose metabolism and Innate Immune System. |
| AMPH | Stiff-Person Syndrome and Limbic Encephalitis |
| APOD | Breast Cyst and Niemann-Pick Disease |
| ARL6IP4 | Infection by Herpes simplex virus (HSVI), may act as a splicing inhibitor of HSVI |
| | pre-mRNA. |
| ASAH1 | Enzyme is overexpressed in multiple human cancers and cancer progression |
| ASGR2 | Asialoglycoprotein receptor may facilitate hepatic infection_by multiple viruses |
| | including hepatitis B |
| ATP5B | Pyelonephritis |
| NCR3LG1 | Immunoregulatory interactions between a Lymphoid and a non-Lymphoid cell and |
| | Innate Immune System. |
| BCL6 | zinc finger transcription factor 1 controls neurogenesis; including the recruitment |
| | of the deacetylase SIRT1 and resulting in an epigenetic silencing leading to |
| | neuronal differentiation. |
| ARL6IP4 | Herpes simplex virus (HSVI), |
| ASAH1 | Regulator of steroidogenesis |
| ASGR2 | Hepatitis B |
| ATF5 | West Nile Fever and |
| | Triple-Receptor Negative |
| | Breast Cancer |
| ATL3 | Neuropathy, Hereditary Sensory, |
| | Type If and Hereditary Sensory And Autonomic Neuropathy Type 1 |
| NCR3LG1 | Selectively expressed on tumor cells. |
| BCL6 | Primary Mediastinal Large B-Cell Lymphoma and Intravascular Large B-Cell |
| | Lymphoma |
| BICD1 | Lissencephaly 1 |
| ARL14EP | Connect MHC class II-containing cytoplasmic vesicles to the actin network |
| CASP3 | Helicobacter Pylori Infection and Fixed Drug Eruption. |
| CEBPD | Macrophage and regulation of genes involved in immune and inflammatory |
| | responses |
| CHN2 | associated with schizophrenia in men |
| CLCN3 | Cystic Fibrosis. |
| CLDN7 | Ductal Carcinoma In Situ and Chromophobe Renal Cell Carcinoma. |
| CLOCK | Circadian |
| COL1A2 | Osteogenesis Imperfecta, Type Iii |
| | and Osteogenesis Imperfecta, |
| | Type Iv. |
| CRABP1 | Teratocarcinoma and Embryonal |
| | Carcinoma. |
| CRABP2 | Embryonal Carcinoma and Basal |
| | Cell Carcinoma. |
| CREM | Oligoarticular Juvenile Idiopathic Arthritis and Female Stress |
| | Incontinence. |
| CRHBP | Autosomal Dominant Nocturnal |
| | Frontal Lobe Epilepsy |
| CRY1 | Circadian |
| CRYAA | Cataract 9, Multiple Types and Cataract Microcornea Syndrome. |
| CRYAB | Myopathy, Myofibrillar, 2 and Cataract 16, Multiple Types. |
| CRYM | Deafness, Autosomal Dominant 40 and Retinitis Pigmentosa 22. |
| CTGF | Limited Scleroderma and Systemic Scleroderma. |
| CTNNB1 | Pilomatrixoma and Neurodevelopmental Disorder With Spastic Diplegia And |
| | Visual Defects. |
| CTNND2 | Benign Adult Familial Myoclonic Epilepsy and Cri-Du-Chat Syndrome. |
| CCKBR | Panic Disorder and Anxiety. |
| DCC | Mirror Movements 1 and |
| | Gaze Palsy, Familial Horizontal, With Progressive Scoliosis 2, With Impaired |
| | Intellectual Development |
| CUX2 | TF; Regulates dendrite development and branching, dendritic spine formation, |
| | and synaptogenesis in cortical layers II-III |
| DDIT4 | Required for mTORC1-mediated defense against viral protein synthesis and virus |
| | replication (By similarity). Inhibits neuronal differentiation and neurite outgrowth |
| | mediated by NGF via its effect on mTORC1 activity. Required for normal neuron |
| | migration |
| | during embryonic brain development. |
| | Plays a role in neuronal cell death. |
| DLX1 | TF; differentiation of interneurons, |
| | such as amacrine and bipolar cells in the developing retina development of the |
| | ventral forebrain |
| DNAJB5 | Master negative regulator of cardiac hypertrophy |
| DPP7 | Innate Immune System. |
| DUSP1 | Amyotrophic Lateral Sclerosis 7 and Amyotrophic Lateral Sclerosis 9. |
| EGR1 | Circadian C2H2-type zinc-finger proteins rhythmic expression of core-clock gene |
| | ARNTL/BMAL1 in the suprachiasmatic nucleus |
| EMX2OS | non-coding RNA class. Diseases associated with EMX2OS include Hypertension, |
| | Essential |
| ENO1 | Hashimoto Encephalopathy and Cancer-Associated Retinopathy. |
| ENO2 | Granular Cell Tumor and Small Cell Carcinoma. |
| ENO3 | Glucose metabolism and HIF-1 signaling pathway. |
| ENPP2 | motility-related processes such as angiogenesis and |
| | neurite outgrowth. Acts as an angiogenic factor |
| EP300 | histone acetyltransferase and regulates transcription HIV-1 infection, it is recruited |
| | by the viral protein Tat Participates in CLOCK or NPAS2-regulated rhythmic gene |
| | transcription; exhibits a circadian association with CLOCK or NPAS2, correlating |
| | with increase in PER1/2 mRNA and histone H3 acetylation on the PER1/2 |
| | promoter |
| ERRFI1 | Progesterone Resistance and Atrial Heart Septal Defect. |
| FABP7 | Establishment of the radial glial fiber in the developing brain; necessary for the |
| | migration of immature neurons to establish cortical layers |
| FAM107A | Brain Cancer. |
| FBXL3 | Circadian; ubiquitin protein ligase complex maintenance of both the |
| | speed and the robustness of the circadian clock oscillation |
| FBXO45 | Control synaptic activity by controlling UNC13A via ubiquitin dependent pathway |
| FIGNL1 | DNA double-strand break repair via homologous recombination |
| FN1 | Glomerulopathy With Fibronectin Deposits 2 and Spondylometaphyseal |
| | Dysplasia, Corner Fracture Type. |
| FUBP1 | MYC; activator and repressor of transcription. |
| GAA | Glycogen Storage Disease Ii and Glycogen Storage Disease. |
| GABBR1 | Brain disorders such as schizophrenia and epilepsy. |
| GABRB1 | Pathogenetics of schizophrenia (GABA) A receptor |
| GABRB3 | Angelman syndrome, Prader-Willi syndrome, nonsyndromic orofacial clefts, |
| | epilepsy and autism |
| GAD1 | Role in the stiff man syndrome. |
| GCLC | Gamma-Glutamylcysteine |
| | Synthetase Deficiency, |
| | Hemolytic Anemia Due To and Myocardial Infarction. |
| GRB2 | Adapter protein that provides a critical link between cell surface growth factor |
| | receptors and the Ras signaling pathway. |
| GRIA1 | (AMPA) receptors |
| GRIK5 | GRIK5 include Schizophrenia Glutamate Ionotropic Receptor Kainate |
| GRIN2A | (NMDA) receptor subunit |
| GRM3 | Schizophreniform Disorder and Schizophrenia; Glutamate Metabotropic |
| | Receptor 3 |
| HDAC4 | Initiation of transcription and translation elongation at the HIV-1 LTR. |
| HDAC5 | Initiation of transcription and translation elongation at the HIV-1 LTR |
| HIF1A | Hypoxia and Retinal Ischemia. |
| HIP1 | Clathrin-mediated endocytosis and trafficking |
| HMOX1 | Heme Oxygenase 1 Deficiency and Pulmonary Disease, Chronic Obstructive. |
| HOMER2 | Regulate group 1 |
| | metabotrophic glutamate receptor |
| HTR2A | Major Depressive Disorder; Obsessive-Compulsive Disorder. |
| HTRA1 | Age-related macular degeneration type 7 |
| ITM2A | Osteo- and chondrogenic differentiation |
| ITPKB | Inositol phosphate metabolism |
| KALRN | Coronary Heart Disease 5 and Keratomalacia. |
| LMX1A | LIM Homeobox Transcription Factor 1 Alpha development of dopamine producing |
| | neurons during embryogenesis. |
| LRCH4 | |
| LYZ | Amyloidosis, Familial Visceral and Al Amyloidosis. |
| MAL | vesicular trafficking cycling between the Golgi complex and the apical plasma |
| | membrane |
| MAOB | oxidation of monoamines such as dopamine, serotonin and adrenalin |
| MPDZ | control of AMPAR potentiation and synaptic plasticity in excitatory synapses; |
| | interact with the HTR2C receptor and may cause it to clump at the cell surface |
| MPZ | Schwann cells of the peripheral nervous system |
| MT1X | Metallothioneins |
| MT2A | Scrapie and Xeroderma Pigmentosum, Complementation Group B. |
| NCOA7 | transcriptional activities |
| | Aryl Hydrocarbon Receptor and AHR Pathway. |
| NDUFS1 | Respiratory electron transport, ATP synthesis by chemiosmotic coupling, and heat |
| | production by uncoupling proteins. |
| NDUFV2 | Mitochondrial Complex I Deficiency, Nuclear Type 7 and Leigh Syndrome With |
| | Leukodystrophy. |
| NEFH | Charcot-Marie-Tooth Disease, Axonal, Type 2Cc and Amyotrophic Lateral |
| | Sclerosis 1. |
| NEFL | Charcot-Marie-Tooth Disease, Demyelinating, Type 1F and Charcot-Marie-Tooth |
| | Disease, Dominant Intermediate G. |
| NET1 | Oppositional Defiant Disorder and Breast Cancer. |
| NFKBIA | Inflammatory responses |
| NPY1R | Body Mass Index Quantitative Trait Locus 11. |
| NR1D1 | Represses the circadian clock transcription factor aryl hydrocarbon receptor |
| | nuclear translocator-like protein 1 (ARNTL) |
| NR3C1 | Glucocorticoid receptor, which can function both as a transcription factor; pituitary |
| | gland |
| NR3C2 | Mineralocorticoid receptors (MRs) are nuclear hormone receptors |
| NRGN | NRGN is a direct target for thyroid hormone in human brain |
| | Hypothyroidism; poor ability to tolerate cold, a feeling of tiredness, constipation, |
| | depression, and weight gain |
| NSF | Tetanus and Neuronal Intranuclear Inclusion Disease. |
| NTRK2 | Glia neutrophin-dependent calcium signaling in glial cells and mediate |
| | communication between neurons and glia. |
| PCP4 | neuronal differentiation through activation of calmodulin-dependent |
| | kinase signaling pathways |
| PDIA3 | Maxillary Sinus Squamous Cell; Carcinoma and Anomalous Left Coronary Artery |
| | From |
| | The Pulmonary Artery. |
| PDK4 | Platelet Glycoprotein Iv Deficiency and Diabetes Mellitus, |
| | Noninsulin-Dependent. |
| PER1 | Circadian |
| PER2 | Circadian |
| PER3 | Circadian |
| BLOC1S6 | Hermansky-Pudlak Syndrome 9 |
| PMP22 | Charcot-Marie-Tooth Disease And Deafness and Charcot-Marie-Tooth Disease, |
| | Demyelinating, Type 1A. |
| PPP1R2 | Cataract 11, Multiple Types and Congenital Stationary Night Blindness. |
| PRKCE | Rift Valley Fever and Streptococcus Pneumonia. |
| PURA | Mental Retardation, Autosomal Dominant 31 |
| RHOU | Signaling by Rho GTPases and Innate Immune System. |
| RNF125 | Tenorio Syndrome. |
| RPS6KA5 | Activated TLR4 signaling and Bladder cancer. |
| RPSA | Asplenia, Isolated Congenital and Venezuelan Equine Encephalitis. |
| SESN1 | Maxillary Cancer. |
| SF3B1 | Myelodysplastic Syndrome and Autosomal Recessive Pyridoxine-Refractory |
| | Sideroblastic Anemia 2. |
| SGK3 | Breast Cancer |
| SLC4A7 | Renal Tubular Acidosis and Usher Syndrome, Type Iiia. |
| SMPD2 | Lipid Storage Disease and Coffin-Siris Syndrome 1. |
| SNAP25 | Myasthenic Syndrome, Congenital, 18 and Presynaptic Congenital Myasthenic |
| | Syndromes. |
| SPTBN2 | Spinocerebellar Ataxia 5 and Spinocerebellar Ataxia, Autosomal Recessive 14. |
| STX1A | Cystic Fibrosis and Osteogenesis Imperfecta, Type Xv. |
| STXBP1 | Early Infantile Epileptic |
| | Encephalopathy. |
| STXBP2 | Hemophagocytic |
| | Lymphohistiocytosis. |
| SV2A | Alcohol-Related Birth Defect |
| SYN2 | Schizophrenia and Bipolar Disorder. |
| TACSTD2 | Carcinoma-associated antigen. Tumor Associated Calcium Signal Transducer 2; |
| TAGLN3 | Actin filament binding. |
| TF | Atransferrinemia and Iron Deficiency Anemia. |
| TFAP2B | AP-2 family of transcription factors |
| TFRC | Immunodeficiency 46 and Combined Immunodeficiency, X-Linked. |
| TPH2 | Serotonin Major Depressive Disorder and Depression. |
| TRIB2 | Wnt/Hedgehog/Notch and DNA Damage; Uveitis and Narcolepsy. |
| TRIP10 | Translocation of GLUT4 to the plasma membrane in response to insulin signaling; |
| | Wiskott-Aldrich Syndrome. |
| TSPAN13 | Regulation of cell development, activation, growth and motility |
| TULP1 | Physiology of photoreceptors Leber Congenital Amaurosis 15 and Retinitis |
| | Pigmentosa 14. |
| WIF1 | Functions to inhibit WNT proteins; tumor suppressor gene, Esophageal Basaloid |
| | Squamous Cell Carcinoma. |
| ZMYM1 | Zinc Finger MYM-Type Containing 1 |
| LMO3 | Cysteine-rich LIM domain Oncogene member |

### Example 7

### Neural Organoids for Testing Drug Efficacy

Neural organoids can be used for pharmaceutical testing, safety, efficacy, and toxicity profiling studies. Specifically, using pharmaceuticals and human neural organoids, beneficial and detrimental genes and pathways associated with drug abuse susceptibility can be elucidated. Neural organoids as provided herein can be used for testing candidate pharmaceutical agents, as well as testing whether any particular pharmaceutical agent inter alia for drug abuse susceptibility should be administered to a particular individual based on responsiveness, alternation, mutation, or changes in gene expression in a neural organoid produced from cells from that individual or in response to administration of a candidate pharmaceutical to said individual's neural organoid.

### Other Embodiments

From the foregoing description, it will be apparent that variations and modifications can be made to the invention described herein to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

Having described the invention in detail and by reference to specific aspects and/or embodiments thereof, it will be apparent that modifications and variations are possible without departing from the scope of the invention defined in the appended claims. More specifically, although some aspects of the present invention may be identified herein as particularly advantageous, it is contemplated that the present invention is not limited to these particular aspects of the invention. Percentages disclosed herein can vary in amount by ±10, 20, or 30% from values disclosed and remain within the scope of the contemplated invention.

APPENDIX
Brain Structure Markers and Accession No.

| Brain Region | Gene Accession |
|---|---|
| Cerebellar | |
| ATOH1, | NM_005172.1 |
| PAX6 | NM_000280.4 |
| SOX2 | NM_003106.3 |
| LHX2 | NM_004789.3 |
| GRID2 | NM_001510.3 |
| Dopaminergic | |
| MAT2 | • NM_003054.4 |
| DAT | NM_001044.4 |
| D2 | NM_000795.3 |
| Cortical | |
| NeuN | NM_001082575.2 |
| FOXP2 | NM_014491.3 |
| CNTN4 | NM_175607.2 |
| TBR1 | NM_004612.3 |
| Retinal | |
| GUY2D | NM_000180.3 |
| GUY2F | NM_001522.2 |
| RAX | NM_013435.2 |
| Granular Neuron | |
| SOX2 | NM_003106.3 |
| NeuroD1 | NM_002500.4 |
| DCX | NM_000555.3 |
| EMX2 | NM_000555.3 |
| FOXG1 | NM_005249.4 |
| PROX1 | NM_001270616.1 |
| Spinal Cord | |
| HOXA1 | NM_005522.4 |
| HOXA2 | NM_006735.3 |
| HOXA3 | NM_030661.4 |
| HOXB4 | NM_024015.4 |
| HOXAS | NM_019102.3 |
| HOSCS | NM_018953.3 |
| HOXDI3 | NM_000523.3 |
| GABAergic | |
| NKCCI | NM_000338.2 |
| KCC2 | NM_001134771.1 |
| Microglia | |
| AIF1 | NM_032955.2 |
| CD4 | NM_000616.4 |
| Brain Stem | |
| FGF8 | NM_033165.3 |
| INSM1 | NM_002196.2 |
| GATA2 | NM_001145661.1 |
| ASCL1 | NM_004316.3 |
| GATA3 | NM_001002295.1 |

What is claimed is:

1. A method for detecting drug abuse susceptibility in a human, using a patient-specific pharmacotherapy, the method comprising:
a) procuring one or a plurality of cell samples from a human, comprising one or a plurality of cell types;
b) reprogramming the one or the plurality of cell samples to produce one or a plurality of induced pluripotent stem cell samples;
c) treating the one or the plurality of induced pluripotent stem cell samples to obtain one or more patient specific neural organoids;
d) collecting a biological sample from the patient specific neural organoid;
e) detecting changes in susceptibility to drug abuse biomarker expression from the patient specific neural organoid sample that are differentially expressed in humans abusing one or a plurality of drugs;
f) performing assays on the patient specific neural organoid to identify therapeutic agents that alter the differentially expressed susceptibility to substance use disorders biomarkers in the patient-specific neural organoid sample; and
g) administering a therapeutic agent for the susceptibility to drug abuse to treat the human.

2. The method of claim 1, wherein the at least one cell sample reprogrammed to the induced pluripotent stem cell is a fibroblast derived from skin or blood cells from humans.

3. The method of claim 2, wherein the fibroblast derived skin or blood cells from humans are identified using the genes identified in Table 1,Table 4, or Table 5.

4. The method of claim 1, wherein the measured biomarkers comprise nucleic acids, proteins, or their metabolites.

5. The method of claim 1, wherein the measured biomarkers comprise one or a plurality of biomarkers identified in Table 1 or Table 4 or variants thereof and can be correlated with susceptibility to drug abuse and progression.

6. The method of claim 5, further wherein a combination of susceptibility to substance use disorders biomarkers is detected, the combination comprising a nucleic acid encoding human NURR1, Lmx1a, Lmx1b, Neurog2, OTX2, Nolz1, and NDNF and associated variants or a plurality of biomarkers comprising a nucleic acid encoding human genes identified in Table 1 or Table 4.

7. The method of claim 1, wherein the neural organoid biological sample is collected after about one hour up to about 12 weeks post inducement.

8. The method of claim 7, wherein the neural organoid sample is procured from structures of the neural organoid that mimic structures developed in utero at about 5 weeks.

9. The method of claim 7, wherein the neural organoid at about twelve weeks post-inducement comprises encoded structures and cell types of retina, cortex, midbrain, hindbrain, brain stem, or spinal cord.

10. The method of claim 7, wherein the neural organoid contains microglia, and one or a plurality of drug use susceptibility biomarkers as identified in Table 1 or Table 4.

11. The method of claim 1, wherein the method is used to detect environmental factors that cause or exacerbate drug abuse susceptibility.

12. The method of claim 1, wherein the method is used in predictive toxicology to detect factors that cause or exacerbate drug abuse or drug abuse disorder susceptibility.

13. The method of claim 1, wherein the method is used to identify causes or accelerators of drug abuse susceptibility.

14. The method of claim 1, wherein the method is used to identify nutritional factors or supplements for treating drug abuse and drug abuse susceptibility.

15. The method of claim 14, wherein the nutritional factor or supplement is for pathways regulated by genes identified in Tables 1 or 4, and administering treatments directed at these pathway targets.

16. A patient-specific pharmacotherapeutic method for reducing the susceptibility to substance use disorders in a human, the method comprising:
a) procuring one or a plurality of cell samples from a human, comprising one or a plurality of cell types;
b) reprogramming the one or the plurality of cell samples to produce one or a plurality of induced pluripotent stem cell samples;
c) treating the one or the plurality of induced pluripotent stem cell samples to obtain one or more patient specific neural organoids;
d) collecting a biological sample from the patient specific neural organoid;
e) detecting biomarkers of the susceptibility to drug abuse in the patient specific neural organoid sample;
f) administering a therapeutic agent for drug abuse to the human.

17. The patient specific pharmacotherapeutic method of claim 16, wherein the measured biomarkers comprise biomarkers identified in Tables 1 or 4.

18. The method of claim 16 further wherein the measured biomarker is a gene encoding nucleic acids, protein, or their metabolite encoding the biomarkers identified in Tables 1 or 4.

19. A plurality of biomarkers comprising a diagnostic panel for predicting a risk for developing substance use disorders, comprising one or a plurality subset of the biomarkers as identified in Tables 1 or 4.

20. The diagnostic panel of claim 19, further wherein the subset of measured biomarkers comprise a gene encoding a nucleic acid, proteins, or their metabolites as identified in Tables 1 or 4.

21. A method of pharmaceutical testing for drug screening, toxicity, safety, and/or pharmaceutical efficacy and studies of novel pharmaceuticals for use in treating drug abuse, using a patient specific neural organoid.

22. A method for detecting at least one biomarker of any of claim 5, 17, 18, 19, or 20, the method comprising:
a) obtaining a biological sample from a human patient; and
b) contacting the biological sample with an array comprising specific-binding molecules for the at least one biomarker and detecting binding between the at least one biomarker and the specific binding molecules.

23. The method of claim 22, wherein the biomarker is a gene therapy target as provided in Table 1 or 5.

24. A kit comprising a gene array of containing one or a plurality of nucleic acid biomarkers of claim 5, 12, 13, 14, or 15 in a human patient.

25. The kit of claim 24 containing a container for collection of a tissue sample from a human.

26. The kit of claim 25 wherein reagents required for RNA isolation from a human tissue sample are included.

27. The kit of claim 24 comprising biomarkers for substance abuse or susceptibility to substance use disorders.

28. A kit, comprising the container of any of the claims 24-27 and a label or instructions for collection of a sample from a human, isolation of cells, inducement of cells to become pluripotent stem cells, growth of patient-specific neural organoids, isolation of RNA, execution of the array and calculation of gene expression change and prediction of concurrent or future susceptibility to drug abuse.

29. The method of claim 22, wherein the biomarkers are genes encoding nucleic acids, proteins, or their metabolites.

30. A method for detecting one or a plurality of biomarkers from different human chromosomes associated with the susceptibility to substance use disorders using data analytics that obviates the need for whole genome sequence analysis of patient genomes.

31. The method of claim 30, wherein the gene expression level changes are used to determine clinically relevant symptoms and treatments.

32. The method of claim 30, wherein the neural organoids are used to identify novel biomarkers that serve as data input for development of algorithm techniques as predictive analytics.

33. The method of claim 30, wherein algorithmic techniques include artificial intelligence, machine and deep learning as predictive analytics tools for identifying biomarkers for diagnostic, therapeutic target and drug development processes.

34. A method for predicting a risk for of drug abuse susceptibility in a human, the method comprising:
a) procuring one or a plurality of cell samples from the human, comprising one or a plurality of cell types;
b) reprogramming the one or the plurality of cell samples to produce one or a plurality of induced pluripotent stem cell samples;
c) treating the one or the plurality of induced pluripotent stem cell samples to obtain a neural organoid;
d) collecting a biological sample from the neural organoid;
e) measuring the susceptibility to drug abuse biomarkers in the neural organoid sample; and
f) detecting changes in susceptibility to drug abuse biomarker expression from the patient specific neural organoid sample that are differentially expressed in humans abusing one or a plurality of addictive drugs.

35. The method of claim 34, wherein the at least one cell sample reprogrammed to the induced pluripotent stem cell is a fibroblast.

36. The method of claim 34, wherein the measured biomarkers comprise nucleic acids, proteins, or their metabolites.

37. The method of claim 34, wherein the measured susceptibility to substance use disorders biomarkers are nucleic acids encoding human NURR1, Lmx1a, Lmx1b, Neurog2, OTX2, Nolz1, and NDNF and associated variants or a plurality of biomarkers comprising a nucleic acid encoding human genes identified in Table 1 or Table 4.

38. The method of claim 34, wherein the measured biomarkers comprise one or a plurality of genes as identified in Tables 1, or 5.

39. The method of claim 34, wherein the neural organoid sample is procured from minutes to hours up to 15 weeks post inducement.

40. The method of claim 1, wherein the biomarkers to be tested are one or a plurality of biomarkers in Table 1 or 4.

41. The method of claim 34, wherein the biomarkers to be tested are one or a plurality of biomarkers in Table 5.

42. A method of using a neural organoid along with confirmatory data, and novel data to develop signature algorithms with machine learning, artificial intelligence and deep learning for drug abuse susceptibility.

43. The method of claim 34, wherein the method is used for diagnostic, therapeutic target discovery and drug action discovery for drug abuse susceptibility and related comorbidities as listed in Table 6.

44. The method of claim 1, 16, or 34, wherein the neural inventive novel organoid data is corroborated in post mortem or biopsy tissues from idiopathic patients and extensively identifies known biomarkers for the susceptibility to drug abuse and comorbidities.

45. The method of claim 44, wherein the method is used with induced pluripotent stem cells from any skin cell, tissue, or organ from the human body allowing for an all-encompassing utility for diagnostics, therapeutic target discovery, and drug development.

46. The method of claim 1, 16, or 34, wherein the method and/or neural organoid is used for guided and patient specific toxicology guided by genes form patient's selective vulnerability to infectious agents or to other environmental factors.

47. The method of claim 1, 16, or 34, wherein the method can be used to identify nutritional and toxicological prenatal care so that the child develops normally in utero.

48. The method of claim 1, wherein the measured biomarkers comprise nucleic acids, proteins, or their metabolites.

49. A method of using neural organoids to obtain human exosomes and their biomarker repertoire for diagnostic and therapeutic purposes of brain diseases in other tissues (blood).

50. The method of claim 1, wherein an individual is susceptible to abusing addictive opioid medications including OxyContin and fentanyl.

51. The method of claim 1, wherein the method is used to identify the risk of developing the comorbidities of cancer, perturbation of circadian rhythms, and neuropsychiatric disorders, including schizophrenia in individuals with drug abuse susceptibility.

52. The method of claim 1, wherein the method is used to identify risk of developing the co-morbidity listed in Table 6, in an individual who is susceptible to drug abuse.

53. The method of claim 1, wherein the method is used to identify novel non-addictive pain medications.
